(19) United States
(12) Patent Application Publication
(10) Pub. No.: US 2015/0182160 A1
(43) Pub. Date: Jul. 2, 2015

(54) FUNCTION OPERATING METHOD BASED ON BIOLOGICAL SIGNALS AND ELECTRONIC DEVICE SUPPORTING THE SAME

(71) Applicant: Samsung Electronics Co., Ltd., Suwon-si (KR)

(72) Inventors: Sang Heon KIM, Gumi-si (KR); Min Chul KIM, Daegu (KR); Tae Hwan WI, Suwon-si (KR); Eun Yeung LEE, Chilgok-gun (KR); Hae Dong LEE, Daegu (KR); Yong Gil HAN, Gumi-si (KR)

(21) Appl. No.: 14/571,887

(22) Filed: Dec. 16, 2014

(30) Foreign Application Priority Data
Dec. 30, 2013 (KR) 10-2013-0166422

(51) Int. Cl. A61B 5/00; A61B 5/0205; A61B 5/11; A61B 7/00; A61B 5/03; A61B 5/0402; A61B 5/0488; A61B 5/02

(52) U.S. Cl. CPC A61B 5/459; A61B 5/0488; A61B 5/0205; A61B 5/02007; A61B 7/006; A61B 5/0082; A61B 5/03; A61B 5/0402; A61B 5/11; A61B 5/4266; A61B 5/742; A61B 5/7475; A61B 5/0006; A61B 5/681; A61B 5/0022; A61B 5/05

(57) ABSTRACT

A biological signal-based function is provided. The biological signal-based function includes collecting, by a first electronic device arranged on a wrist, biological signals about a state of biological tissue using an Electromyogram (EMG) sensor and an auxiliary sensor, and outputting information related to the collected biological signals to a first display module of the first electronic device.

### CROSS-REFERENCE TO RELATED APPLICATION(S)

This application claims the benefit under 35 U.S.C §119(a) of a Korean patent application filed on Dec. 30, 2013 in the Korean Intellectual Property Office and assigned Serial number 10-2013-0166422, the entire disclosure of which is hereby incorporated by reference.

### TECHNICAL FIELD

The present disclosure relates to operation of an electronic device based on biological signals. More particularly, the present disclosure relates to a method and a device for operating electronic device functions based on biological signals in order to support various electronic device functions by collecting biological signals of a user based on a watch-type electronic device.

### BACKGROUND

Electronic devices of the related art, such as desktop devices, are non-portable. Such electronic devices have recently evolved into mobile devices, and have further evolved into wearable devices. For example, a watch-type electronic device is worn by a certain body portion of a user so as to allow the user to more easily control the electronic device at a desired time.

However, wearable electronic devices of the related art which have simple computing functions are limited in terms of utility.

Therefore, a need exists for a method and a device for operating electronic device functions based on biological signals in order to support various electronic device functions by collecting biological signals of a user based on a watch-type electronic device.

The above information is presented as background information only to assist with an understanding of the present disclosure. No determination has been made, and no assertion is made, as to whether any of the above might be applicable as prior art with regard to the present disclosure.

### SUMMARY

Aspects of the present disclosure are to address at least the above-mentioned problems and/or disadvantages and to provide at least the advantages described below. Accordingly, an aspect of the present disclosure is to provide a method and a device for operating electronic device functions based on biological signals in order to support various electronic device functions by collecting biological signals of a user based on a watch-type electronic device.

In accordance with an aspect of the present disclosure, a method for operating an electronic device is provided. The method includes collecting, by a first electronic device arranged on a wrist, biological signals about a state of biological tissue using an Electromyogram (EMG) sensor and an auxiliary sensor, and outputting information related to the collected biological signals to a first display module of the first electronic device.

In accordance with another aspect of the present disclosure, an electronic device is provided. The electronic device includes a first sensor module including an EMG sensor configured to detect a state of a muscle of a wrist and an auxiliary sensor configured to detect biological signals about a state of biological tissue of the wrist, a first display module configured to output information related to the biological signals collected by the first sensor module, and a first device control module configured to control the first sensor module and the output of the information.

In accordance with another aspect of the present disclosure, an electronic device is provided. The electronic device includes a second communication module configured to receive biological signals collected by a first sensor module including an EMG sensor and an auxiliary sensor configured to detect the biological signals about a state of biological tissue of a wrist, a second display module configured to output service information related to the received biological signals, and a second device control module configured to control the collection of the biological signals and the output of the service information.

Other aspects, advantages, and salient features of the disclosure will become apparent to those skilled in the art from the following detailed description, which, taken in conjunction with the annexed drawings, discloses various embodiments of the present disclosure.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a diagram schematically illustrating a system for supporting functions based on biological signals according to an embodiment of the present disclosure;

FIG. 2 is a diagram illustrating a first electronic device for supporting operation of functions based on biological signals according to an embodiment of the present disclosure;

FIG. 3 is a schematic diagram illustrating an outward appearance of a first electronic device according to an embodiment of the present disclosure;

FIG. 4A is a schematic diagram illustrating a first electronic device according to an embodiment of the present disclosure;

FIG. 4B is a schematic diagram illustrating a first electronic device according to an embodiment of the present disclosure;

FIG. 5 is a block diagram illustrating a second electronic device according to an embodiment of the present disclosure;

FIG. 6 is a diagram illustrating a second device control module according to an embodiment of the present disclosure;

FIG. 7 is a diagram illustrating a function operation layer of a second device control module according to an embodiment of the present disclosure;

FIG. 8 is a diagram illustrating an ontology module according to an embodiment of the present disclosure;

FIG. 9 is a flow diagram illustrating communication between a first electronic device and a second electronic device according to an embodiment of the present disclosure;

FIG. 10 is a diagram illustrating a biological signal-based service according to an embodiment of the present disclosure;

FIG. 11 is a diagram illustrating an operation of a biological signal-based device according to an embodiment of the present disclosure;

FIG. 12 is a diagram illustrating an operation of a biological signal-based service according to an embodiment of the present disclosure;

FIG. 13 is a diagram illustrating a screen interface provided during provision of a biological signal-based service according to an embodiment of the present disclosure;

FIG. 14 is a diagram illustrating a screen interface provided during provision of a biological signal-based service according to an embodiment of the present disclosure;

FIG. 15 is a diagram illustrating a screen interface provided during provision of a biological signal-based service according to an embodiment of the present disclosure;

FIG. 16 is a diagram illustrating an operation of a biological signal-based device according to an embodiment of the present disclosure;

FIG. 17 is a diagram illustrating an operation of a biological signal-based device according to an embodiment of the present disclosure;

FIG. 18 is a flowchart illustrating a method of operating a first electronic device according to an embodiment of the present disclosure; and

FIG. 19 is a diagram illustrating a method of operating a second electronic device according to an embodiment of the present disclosure.

Throughout the drawings, it should be noted that like reference numbers are used to depict the same or similar elements, features, and structures.

### DETAILED DESCRIPTION

The following description with reference to the accompanying drawings is provided to assist in a comprehensive understanding of various embodiments of the present disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the present disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the present disclosure. Accordingly, it should be apparent to those skilled in the art that the following description of various embodiments of the present disclosure is provided for illustration purpose only and not for the purpose of limiting the present disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to skill in the art, may occur in amounts that do not preclude the effect the characteristic was intended to provide.

The term “include,” “comprise,” “including,” or “comprising” used herein indicates disclosed functions, operations, or existence of elements but does not exclude other functions, operations or elements. It will be further understood that the terms “comprises”, “comprising,”, “includes” and/or “including”, when used herein, specify the presence of stated features, integers, operations, elements, and/or components, but do not preclude the presence or addition of one or more other features, integers, operations, elements, components, and/or groups thereof.

The meaning of the term “or” used herein includes any combination of the words connected by the term “or”. For example, the expression “A or B” may indicate A, B, or both A and B.

The terms, such as “first”, “second”, and the like, used herein may refer to various elements of various embodiments, but do not limit the elements. For example, such terms do not limit the order and/or priority of the elements. Furthermore, such terms may be used to distinguish one element from another element. For example, “a first user device” and “a second user device” indicate different user devices. For instance, without departing the scope of the present disclosure, a first element may be named as a second element, and similarly, a second element may be named as a first element.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, and the like).

The terminology used herein is not for delimiting the present disclosure but for describing specific various embodiments. The terms of a singular form may include plural forms unless otherwise specified.

The terms used herein, including technical or scientific terms, have the same meanings as understood by those skilled in the art. It will be further understood that terms in common usage should also be interpreted as is customary in the relevant art and not in an idealized or overly formal detect unless expressly so defined herein.

An electronic device according to the present disclosure may include a communication function. For example, the electronic devices may include at least one of smartphones, tablet Personal Computers (PCs), mobile phones, video telephones, electronic book readers, desktop PCs, laptop PCs, network computers, Personal Digital Assistants (PDAs), Portable Multimedia Players (PMPs), MP3 players, mobile medical devices, cameras, wearable devices (e.g., Head-Mounted-Devices (HMDs), such as electronic glasses), electronic apparel, electronic bracelets, electronic necklaces, electronic accessories, electronic tattoos, and smart watches.

According to some embodiments of the present disclosure, electronic devices may be smart home appliances having communication functions. The smart home appliances may include at least one of, for example, TVs, DVD players, audios, refrigerators, air conditioners, cleaners, ovens, microwave ovens, washing machines, air cleaners, set-top boxes, TV boxes (e.g., Samsung HomeSync™, Apple TV™, or Google TV™), game consoles, electronic dictionaries, electronic keys, camcorders, and electronic picture frames.

According to some embodiments of the present disclosure, electronic devices may include at least one of medical devices (e.g., Magnetic Resonance Angiography (MRA), Magnetic Resonance Imaging (MRI), Computed Tomography (CT), scanners, and ultrasonic devices), navigation devices, Global Positioning System (GPS) receivers, Event Data Recorders (EDRs), Flight Data Recorders (FDRs), vehicle infotainment devices, electronic equipment for vessels (e.g., navigation systems and gyrocompasses), avionics, security devices, head units for vehicles, industrial or home robots, Automatic Teller's Machines (ATMs), and Points Of Sales (POSs).

According to some embodiments of the present disclosure, electronic devices may include at least one of parts of furniture or buildings/structures having communication functions, electronic boards, electronic signature receiving devices, projectors, and measuring instruments (e.g., water meters, electricity meters, gas meters, and wave meters). Electronic devices according to the present disclosure may be one or more combinations of the above-mentioned devices. Furthermore, electronic devices according to the present disclosure may be flexible devices. In addition, it would be obvious to those skilled in the art that electronic devices according to the present disclosure are not limited to the above-mentioned devices.

Hereinafter, electronic devices according to various embodiments of the present disclosure will be described with reference to the accompanying drawings. The term “user” used herein may refer to a person who uses an electronic device or may refer to a device (e.g., an artificial electronic device) that uses an electronic device.

According to a system and method for supporting functions based on biological signals and a device supporting the same, according to various embodiments of the present disclosure, a first electronic device may be, e.g., a wrist-wearable device, for collecting biological signals. The biological signals of the first electronic device may be used alone or in association with other electronic devices so as to provide various services.

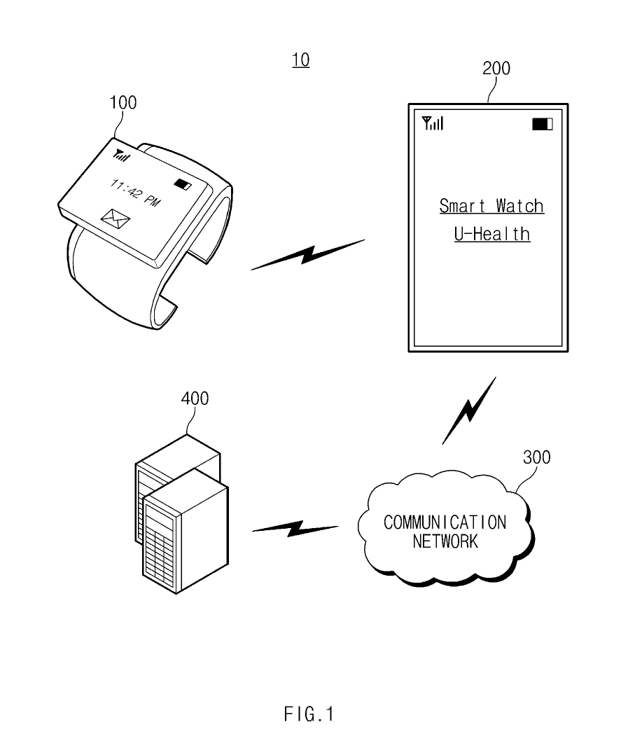

FIG. 1 is a diagram schematically illustrating a system for supporting functions based on biological signals according to an embodiment of the present disclosure.

Referring to FIG. 1, a system 10 for supporting functions based on biological signals may include a first electronic device 100, a second electronic device 200, a communication network 300 and a server device 400.

In the system 10 for supporting functions based on biological signals, the first electronic device 100 may be worn by a wrist so that biological signals of a user may be collected. The collected biological signals may be processed in the first electronic device 100 so as to support a specific function. Furthermore, the collected biological signals may be transferred to the second electronic device 200 so as to support a specific function of the second electronic device 200 linked to the first electronic device 100. In the system 10 for supporting functions based on biological signals, biological signal-related service information provided by the server device 400 through the communication network 300 may be processed in the first electronic device 100 or the second electronic device 200 so as to support a specific service function.

The first electronic device 100 may include an Electromyogram (EMG) sensor and an auxiliary sensor to more clearly detect a biological signal from a wrist. Here, an EMG sensor and an optical sensor, an EMG sensor and an acoustic vibration sensor, or an EMG sensor and an acceleration sensor may be operated together so as to more clearly detect biological signals corresponding to motion of extensor digitorum or extensor digiti minimi which is not easily detectable by an EMG sensor alone. Accordingly, the first electronic device 100 may collect more precise and clear biological signals related to hand motion, and may support various functions based on the collected biological signals. Furthermore, the first electronic device 100 may be tightly fit to a wrist so that the biological signals related to hand motion may be more precisely and clearly detected.

The second electronic device 200 may establish a communication channel to the first electronic device 100 to receive the biological signals collected by the first electronic device 100. The second electronic device 200 may support various functions in response to the received biological signals. For example, the second electronic device 200 may run an application related to the received biological signals, or may receive service information related to the biological signals from the server device 400. The second electronic device 200 may output a screen of the run application or may output the biological signal-related service information through a display unit. The electronic device 200 may transmit, to the first electronic device 100, at least one of activated application screen information and the biological signal-related service information. In connection with collecting the biological signal-related service information, the second electronic device 200 may establish a communication channel to the service device 400 through the communication network 300, and may receive the biological signal-related service information from the server device 400.

The communication network 300 may include a network element for establishing a communication channel between the second electronic device 200 and the server device 400. The communication network 300 may include at least one of various networks, such as a mobile communication network, a 3G telecommunication network and a 4G telecommunication network. The communication network 300 may establish a communication channel between the first electronic device 100 and the second electronic device 200 or a communication channel between the first electronic device 100 and the server device 400 according to the characteristics or type of a communication module of the first electronic device 100. The communication network 300 may transfer the biological signal-related service information provided by the server device 400 to the first electronic device 100 or the second electronic device 200. The communication network 300 may transfer, to the first electronic device 100, the biological signal-related service information stored in the second electronic device 200 or information on various functions operated in the second electronic device 200.

The server device 400 may store various pieces of biological signal-related service information in connection with biological signals. The server device 400 may provide the biological signal-related service information to the first electronic device 100 or the second electronic device 200 through the communication network 300. In an embodiment of the present disclosure, the server device 400 may receive, from the first or second electronic device 100 or 200, the biological signals received by the first electronic device 100, and may provide the biological signal-related service information.

For example, the server device 400 may provide information associated with a Context-Aware-Based U-health Environment Information Service (CAUEIS) or may support the CAUEIS. To provide the CAUEIS, a certain service or a service corresponding to occurrence of a specific event may be provided through context awareness. For example, according to the CAUEIS, a situation of a user is analyzed through internal information or external information, and a specific function may be provided based on the biological signal-related service information associated with a residential area environment of the user. The context awareness may be performed through an inference process of an inference engine predesigned based on at least one of internal information corresponding to the biological signals collected by the first electronic device 100, external information related to an external environment of the first electronic device 100, or the biological signal-related service information provided from the server device 400. In an embodiment of the present disclosure, the system 10 for supporting functions based on biological signals may infer an environment information service suitable for a user's situation instead of simply providing a function. Accordingly, the system 10 for supporting functions based on biological signals may provide a meaningful service based on a result inferred using various pieces of context information instead of simply monitoring a health status. The biological signal-related service information will be described in connection with various embodiments of the present disclosure.

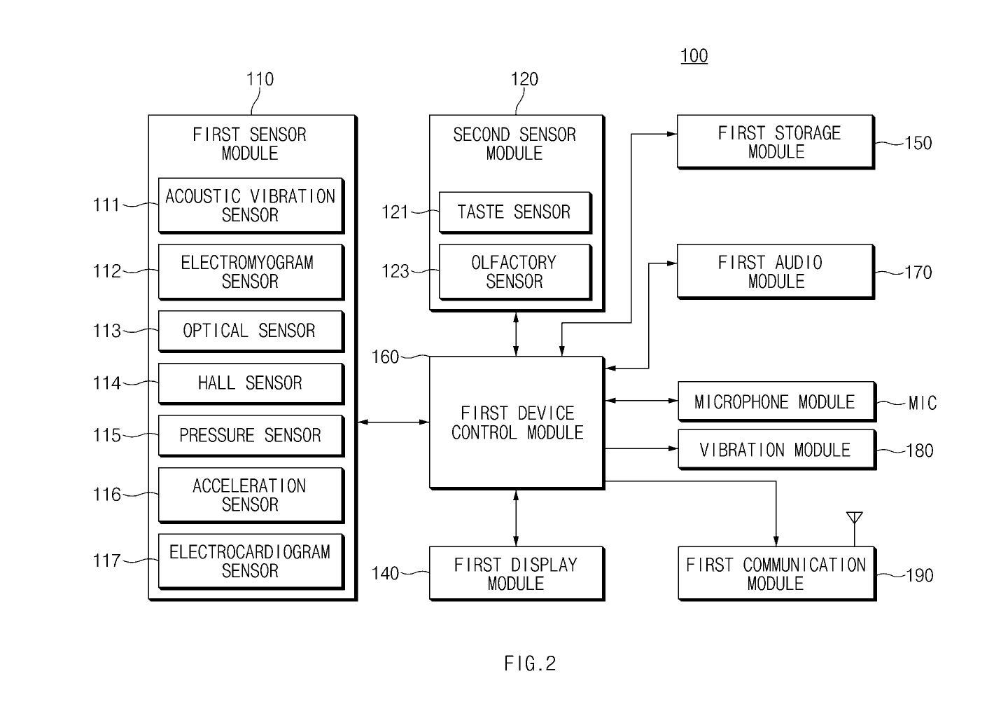

FIG. 2 is a diagram illustrating a first electronic device for supporting operation of functions based on biological signals according to an embodiment of the present disclosure.

Referring to FIG. 2, the first electronic device 100 may include a first sensor module 110, a second sensor module 120, a first display module 140, a first storage module 150, a first audio module 170, a vibration module 180, a first communication module 190, and a first device control module 160.

The detection sensitivity of the first electronic device may be increased by a combination of an EMG sensor 112 and an optical sensor 113, a combination of the EMG sensor 112 and a hall sensor 114, a combination of the EMG sensor 112 and an acceleration sensor 116, a combination of the EMG sensor 112 and an Electrocardiogram (ECG) sensor 117, and a combination of the EMG sensor 112 or an acoustic vibration sensor 111 included in the first sensor module. At least one of the acoustic vibration sensor 111, the optical sensor 113, the hall sensor 114, the acceleration sensor 116, or the ECG sensor 117 may serve as an auxiliary sensor for collecting biological signals.

The first sensor module 111 may include at least one of the acoustic vibration sensor 111, the EMG sensor 112, the optical sensor 113, the hall sensor 114, a pressure sensor 115, the acceleration sensor 116, or the ECG sensor 117. Various pieces of detection information collected by the first sensor module 110 may be provided to the first device control module 160.

The acoustic vibration sensor 111 may include at least one of a piezo sensor or microphone, a laser distance sensor, an accelerometer, or a Mechano-Myography (MMG) sensor. The MMG sensor may noninvasively measure a micro vibration caused by volume change of muscular fibers which occurs when a muscle is activated. The MMG sensor may support signal collection for measuring muscle fatigue and detecting a purpose of motion. The MMG sensor having a small size may perform measurement on fine muscle. Living body portions may generate unique sounds due to a difference of bone density or muscle thickness. The MMG sensor may recognize a difference of sounds so as to detect, e.g., a position of motion of a finger. The MMG sensor may determine a reaction by analyzing a mechanical vibration due to the position. According to an embodiment of the present disclosure, when a finger touches a certain object, a micro sound signal is generated in a human body. The wavelength of this signal varies while the signal spreads through a skin surface, arm muscle and bones and then passes through muscle tissue and hard joints. The MMG sensor may calculate differences of bone density and muscle mass so as to trace back and detect a location where the sound has been originally generated.

According to an embodiment of the present disclosure, muscular contraction causes a micro vibration in a body surface of active muscle. The vibration may be caused by pressure waves generated due to lateral expansion or deformation of muscular fibers of a contracted muscle. The MMG sensor may record and analyze a micro vibration of a low-frequency band (for example, less than about 100 Hz) so as to estimate a muscle action. The MMG sensor may indicate a mechanical state of an active muscle, whereas the EMG sensor may indicate an electrical state of the active muscle. The MMG sensor detects biological signals in connection with mechanical contraction of muscular fibers, and thus may support determination of a mechanical aspect with respect to a muscle action strategy, such as motor unit recruitment or a firing rate.

According to various embodiments of the present disclosure, the acoustic vibration sensor 111 may include a sensor that detects a tremor based on a vibration measured from a surface of a human body. The tremor may be classified into a rest tremor and an action tremor according to whether significant muscle action occurs. Similarly to a muscle vibration, a Force Tremor (FT) occurs when a muscle contracts, and may occur both at a frequency domain of about 3-6 Hz related to inertia of a human body and a stretch reflex and a frequency domain of about 8-12 Hz related to rhythmic input from central nerves to motor neurons. Therefore, in the signals detected by the acoustic vibration sensors 111, FT waves may be mixed with MMG waves for measuring a low-frequency vibration due to lateral deformation of muscular fibers. The FT waves which are a type of a physiological artifact may support detection of detecting signals related to motion of an antagonistic muscle rather than an agonistic muscle with respect to muscular antagonism. When muscular activity increases with respect to vibration characteristics of muscular tissue, an attenuation coefficients of soft tissue increases, thereby attenuating resonance. Therefore, the FT may affect an antagonistic muscle more strongly than an agonistic muscle.

The acoustic vibration sensor 111 may further include at least one of a condenser microphone MIC or an acceleration sensor ACC. In the condenser microphone, an air chamber is attached to a sensor so as to measure an air pressure change of the air chamber due to a vibration of a body surface. The acceleration sensor 116 may be affected by a FT more strongly than the condenser microphone. When fatigue muscle contraction occurs, the acoustic vibration sensor 111 may compare an antagonistic muscle with an antagonistic muscle to compare effects of a FT on MMGs measured by the condenser microphone MIC and the acceleration sensor ACC, thereby supporting calculation of a degree of fatigue.

The EMG sensor 112, in which an amplifier and various filters and measurement units are integrated, induces a surface EMG using two silver electrodes attached in parallel. The silver electrodes which include high-purity silver, e.g., 99.9% pure silver, facilitate an ion flow on a skin surface and enables normal measurement even though a special treatment is applied on a skin before attaching the electrodes thereto.

The EMG sensor 112 may perform EMG measurement on the peripheries of radial arteries and ulnar arteries. The EMG sensor 112 may obtain an EMG from a forearm extensor muscle portion. To perform EMG detection on the forearm extensor muscle portion, the EMG sensor 112 may be attached between extensor digitorum and extensor digiti minimi of a forearm extensor muscle involved in motion of a finger and signals involved in the finger motion may be measured. The EMG sensor 112 is capable of performing EMG measurement with respect to bending of a wrist in a direction of a thumb or in a direction of a little finger.

The EMG sensor 112 may include a muscle stiffness sensor. The EMG sensor 112 may measure muscle activity to be applied to an exoskeleton device for rehabilitation assistance and a muscle strengthening training device. The muscle stiffness sensor may include a multi pressure sensor to measure stiffness of a muscle which varies with muscle activity. The muscle stiffness sensor may include a circular disk and a tip. The muscle stiffness sensor may be fixed to a measurement position using a fixing member, e.g., a belt, in order to measure biological signals. The first device control module 160 may estimate muscle stiffness based on differences among a muscle pressure (FB) measured through the tip of the muscle stiffness sensor, a pressure (FD) of a peripheral muscle contacting with the disk and a pressure applied by the fixing belt. The muscle stiffness sensor employs a relatively simple measurement method, and does not need to directly contact with a skin and thus may be worn on clothes or other materials.

The EMG sensor 112 may include a muscle fiber expansion sensor. The muscle fiber expansion sensor may be a sensor for driving an Exoskeleton for Patients (EXPOS). The muscle fiber expansion sensor which calculates a torque of a joint may include a pneumatic sensor for measuring contraction/expansion of a muscle when a joint moves. Similarly to the muscle stiffness sensor, the muscle fiber expansion sensor may employ a simple measurement method and may be worn on clothes.

The optical sensor 113 optically measures a muscle activity. When light is noninvasively irradiated to a skin, a part of the light is transmitted by skin tissue, such as a skin, subcutaneous fat, and a muscle, and another part of the light is reflected. Optical characteristics of the skin tissue excepting a muscle do not vary with a motion of a human body, but optical characteristics of a muscle vary according to the above-mentioned principle. Therefore, the activity of a muscle may be measured using an amount of reflected light. In order to further describe this operation, the activation of a muscle will be described as follows. A neural signal containing motion-related information is transferred from a brain to a motion-related muscle through a central nervous system so as to stimulate the muscle. The muscle activated by the stimulation is contracted since actin of an I-band included in a muscular fiber and myosin of an A-band pull each other, and thus, the entire muscular fiber bundle becomes short and thick. Therefore, due to concentration of muscular fibers, the muscle has a higher density and stiffness. This phenomenon also brings about a change of an optical characteristic, and, due to a density difference of a muscle, the reflectivity and transmittance of light are changed. Furthermore, a part of light irradiated to a skin surface is transmitted or absorbed due to optical characteristics of each epidermis, muscle, blood vessel and bone, and another part of the light is reflected by each medium. The light reflected by each medium is obtained through a light-receiving unit that is contiguous to a light-emitting unit. Here, the optical characteristics of epidermal tissue or bone tissue do not vary with a motion of a human body, and, in the case of vascular tissue, a weak AC signal is generated according to a heartbeat. However, as described above, the optical characteristic, e.g., the reflectivity, of muscular tissue is changed due to variations of the density and thickness of a muscle caused by human body motion, and thus, an amount of light obtained by the light-receiving unit is changed. Due to such a change, a magnitude of a signal detected by the optical sensor 113 increases when a muscle contracts, and the signal magnitude decreases when the muscle relaxes.

The optical sensor 113 detects detection values using characteristics, such as muscle motion, bone motion, light transmission or reflection of hemoglobin, and the like. The optical sensor 113 may support recognition of finger motion using a change of a finger flexor tendon.

The pressure sensor 115 detects a pressure change according to arrangement of the wrist-wearable first electronic device 100. When the device is worn on fingers including an index finger, middle finger, ring finger and little finger, the device may support recognition of motions of the fingers by measuring extensor digitorum and extensor digiti minimi. The pressure sensor 115 may include an additionally arranged sensor for detecting a motion of a thumb.

The pressure sensor 115 detects changes of extension and flexion from a wrist muscle (side of the back of a hand: extensor, side of the palm: flexor). In order to detect changes of muscles of a wrist and fingers, the pressure sensor 115 may detect pressure changes of flexor digitorum superficialis, flexor digitorum profundus, extensor digitorum, extensor indicis, extensor digiti minimi, flexor hallucis longus, abductor pollicis longus, extensor hallucis longus muscle, and extensor pollicis brevis. The optical sensor 113 may perform detection on the above-mentioned muscles in order to detect a change of a muscle of a wrist or finger.

The optical sensor 113 may include a pulse sensor. The pulse sensor may include a high-brightness LED. The pulse sensor supports pulse measurement and oxygen saturation measurement. The pulse sensor includes a pulse oximeter sensor. The pulse oximeter sensor supports pulse measurement and oxygen saturation measurement using photoelectric Plethysmography (PPG). The pulse sensor supports nonvascular measurement of oxygen saturation of blood in such a manner that lights with different wavelengths of a semiconductor device contact with a finger tip. Oxidized Haemoglobin (HbO₂) and reduced Haemoglobin (Hb) in blood show different spectrum characteristics when being irradiated with light with a wavelength of about 500 nm to 1000 nm. HbO₂ and Hb assume different aspects in terms of light absorptance, and an amount of blood flowing to a finger through an artery varies with a heartbeat. A photodiode arranged in a light-receiving unit of the pulse sensor measures an output voltage changed in proportion to an amount of incident light which varies with amounts of HbO₂ and Hb proportional to a blood amount, so as to support pulse measurement. Furthermore, the pulse sensor supports measurement of oxygen saturation (SaO₂ or SpO₂) according to a change of an amount of light.

The hall sensor 114 may support measurement of blood pressure and pulse. The hall sensor 114 may be arranged on a radial projection or ulnar projection of a wrist (bone projecting from a lower part of a wrist) so as to measure a pulse wave of a radial artery. The hall sensor 114 may include a semiconductor including Indium Antimonide (InSb), Indium Arsenide (InAs), Germanium (Ge) and Silicon (Si). The hall sensor 114 may measure an intensity of a magnetic field using hall electromotive force caused by the magnetic field, and thus may measure a magnetic field of, e.g., a small portion, and may measure a location of a magnetic field if a change of the magnetic field is caused by the location thereof. The hall sensor 114 may obtain a voltage change caused by a location change of a permanent magnet positioned on a radial artery, in the form of an electric signal, by using a magnetic hall device. The electric signal represents a pulse waveform signal. By differentiating such signals through hardware of a circuit, a signal depending on a magnetic field change may be obtained.

A chemical receptor located in an aortic body which is one of sensory nerves for adjusting blood pressure detects an increase of CO₂ and a decrease of O₂ and increases an amount of blood to facilitate the supply of oxygen, causing an increase of blood pressure. A heart rate is increased to increase a supply amount of blood, and thus, a pulse rate is also increases, causing the increase of blood pressure. The hall sensor 114 supports distinguishing by a pulse waveform based on a phenomenon of a separation time difference between a main pulse wave of a radial artery going out of a heart and a wave reflected from an abdominal artery toward the radial artery (both the waves are combined in the case of high blood pressure). Furthermore, personal vascular characteristics may be detected through the above-mentioned phenomenon, and detection of a general characteristic of blood pressure is supported.

The acceleration sensor 116 may include a tri-axial linear acceleration sensor (gravity removal). The acceleration sensor 116 supports motion measurement with respect to a wrist twist and hand motion. An accelerometer which is a sensor for measuring an acceleration of a determined direction may obtain corresponding acceleration information depending on a motion of a user for moving a device. For example, the tri-axial linear acceleration sensor may be installed in a mobile device in the form of a Micro Electro Mechanical System (MEMS) chip so as to recognize a gesture or motion of a user. Since detection information of the acceleration sensor 116 is consecutively input, segmentation may be necessary. To this end, a sliding window technique may be used as a segmentation technique of the acceleration sensor 116. For example, an acceleration average, an absolute average, a standard deviation, energy, covariance, an amount of acceleration change between adjacent points, and a difference of acceleration directions between a start point and an end point of a window are calculated from each window so as to extract 21 features (3 axes×7 types).

The ECG sensor 117 may induce a small potential difference caused by an action of a heart at an appropriate portion on a body surface by using a certain method, and may amplify and record the potential difference. A heart, which acts like a pump for circulating blood throughout a body, regularly repeats contraction and expansion. The pumping action of a heart is performed by virtue of contraction of myocardium. Whenever a heat beats, weak electricity is generated, and thus, a current having a certain intensity flows in a body. The ECG sensor 117 may detect a regular potential generated on a body surface by virtue of the above-mentioned current.

The second sensor module 120 may include at least one of a taste sensor 121 and an olfactory sensor 123. After the first electronic device 100 is arranged on a wrist of a user, the taste sensor 121 may detect sweat generated from the wrist. The olfactory sensor 123 may detect and quantify specific gas in the air. Detection information collected by the second sensor module 120 may be provided to the first device control module 160.

The first display module 140 may display various screens related to the first electronic device 100. For example, the first display module 140 may output a standby screen and a specific function screen of the first electronic device 100. According to an embodiment of the present disclosure, the first display module 140 may output a screen corresponding to sensor enablement of at least one of the first sensor module 110 and the second sensor module 120. The first display module 140 may output various pieces of information related to detected information. The first display module 140 may display a state of connection to the second electronic device 200 and various pieces of information provided by the second electronic device 200. Furthermore, the first display module 140 may output the biological signal-related service information provided by the server device 400 through the second electronic device 200.

The first storage module 150 may store data required for operating the first electronic device 100. For example, the first storage module 150 may store an operating system of the first electronic device 100. Furthermore, the first storage module 150 may store a sensor operating program for operating at least one sensor included in the first and second sensor modules 110 and 120. The first storage module 150 may store a program related to processing of various pieces of detection information collected by the first and second sensor modules 110 and 120. According to an embodiment of the present disclosure, the first storage module 150 may store a program for operating the EMG sensor 112, the acoustic vibration sensor 111, the optical sensor 113, the pressure sensor 115, the hall sensor 114, the acceleration sensor 116, and the ECG sensor 117. Furthermore, the first storage module 150 may store a program for improving the accuracy of biological signals by complexly operating EMG detection information, acoustic vibration detection information, optical detection information, pressure detection information, and acceleration detection information.

The first audio module 170 may support at least one of a function of processing an audio signal output through the first electronic device 100 or a function of collecting audio signals. For example, the first audio module 170 may output an audio signal related to enablement of at least one sensor included in the first and second sensor modules 110 and 120, information for informing of biological signals collected according to the sensor activation, and information for informing of biological signal-related service information.

The vibration module 180 may be an information output unit of the first electronic device 100. For example, the vibration module 180 may output a vibration pattern corresponding to enablement of at least one sensor of the first and second sensor modules 110 and 120, a vibration pattern related to collection of biological signals, and a vibration pattern related to output of biological signal-related service information. According to an embodiment of the present disclosure, the vibration module 180 may implement a vibration having a certain pattern during a process of collecting biological signals by the acoustic vibration sensor 111 or EMG sensor 112. According to an embodiment of the present disclosure, the vibration module 180 may output a vibration pattern for informing of the reception of the biological signal-related service information from the second electronic device 200.

The first communication module 190 may support a communication function of the first electronic device 100. The first communication module 190 may establish, e.g., a communication channel to the second electronic device 200. Alternatively, the first communication module 190 may establish a communication channel to the server device 400. In an embodiment of the present disclosure, the first communication module 190 may include a communication interface for accessing the communication network 300. The first communication module 190 may transfer collected biological signals to the second electronic device 200. The first communication module 190 may receive the biological signal-related service information from the second electronic device 200.

The first device control module 160 may process and transfer various signals related to operation of the first electronic device 100 and may process and transfer data. For example, the first device control module 160 may control operation of at least one of the first sensor module 110 and the second sensor module 120. The first device control module 160 may support output of an icon or menu item related to enablement of at least one sensor or output of an icon or menu item related to activation of a function based on at least one sensor. When an event related to specific sensor enablement or function activation occurs, the first device control module 160 may activate a corresponding sensor. According to an embodiment of the present disclosure, the first device control module 160 may perform a control operation so as to complexly operate the EMG sensor 112 and the acoustic vibration sensor 111, the EMG sensor 112 and the optical sensor 113, the EMG sensor 112 and the hall sensor 114, the EMG sensor 112 and the pressure sensor 115, or the EMG sensor 112 and the ECG sensor 117. The first device control module 160 may improve the accuracy of biological signals by complexly managing detected pieces of information. The first device control module 160 may perform output of specific biological signal-related service information based on obtained detected pieces of information. During this process, the first device control module 160 may receive the biological signal-related service information from the second electronic device 200, or may receive the biological signal-related service information from the server device 400 through the second electronic device 200.

According to an embodiment of the present disclosure, the first device control module 160 may perform surface EMG hand motion modeling. The first device control module 160 may perform surface EMG measurement, noise removal using preprocessing, signal analysis for each action for extracting a hand motion, detail coefficient separation using multi-resolution analysis, hand motion pattern modeling using energy evaluation of the detail coefficient, and hand motion recognition using neural network learning. During this process, the first device control module 160 may perform hand motion recognition through signal analysis based on multi-resolution discrete wavelet analysis.

Since an energy change amount of a signal to be measured is able to be observed and a difference between EMG signals for different motions is not large, an amount of information for distinguishing a plurality of action patterns from single channel signals is so small that it is difficult to distinguish actions through one-dimensional analysis of signals. Therefore, the first device control module 160 obtains information for distinguishing different action patterns from signals of a single channel by using a multi-resolution wavelet. Regarding the multi-resolution wavelet, Daubechies 4 wavelet (db4) is used as a mother wavelet. The Daubechies wavelet is more compact and has excellent performance for irregular-shaped signals in comparison with another wavelet transform, and thus may enable more efficient analysis of acoustic emission signals.

As described above, the first electronic device 100 may perform collection of biological signals and may support various functions related to the biological signals. For example, the first electronic device 100 may support at least one of a biometric U-health function, an active oxygen measurement function, a blood pressure detection function, a low blood pressure notifying function, a function of detecting and notifying signals of a brain hemorrhage and myocardial infarction, a blood vessel measurement function, a pulse wave stress measurement function, a medicine taking assistant function, a fall detection function, an action detection function, a heart rate measurement function, a living-alone senior monitoring function, a snoring prevention function, a recipe providing function, a safe driving assistant or drowsy driving prevention function, a function of detecting an abnormal environment state using an olfactory sensor, a function of measuring sweat, moisture or secretion using a taste sensor, a business card recognition function (for example, be performed based on a Personal Area Network (PAN)), a bone conduction phone function, an anti-static function, a CAUEIS function, a biometric based emotion recognition service function, a Parkinson's disease diagnosis function, a lie detection function, a function of recognizing tennis swing using an acceleration sensor, a fieldwork function, a home network function, a U-themepark service function, a ComMotion function, a shopping assistant function, a cyber-guide function, a conference assistant function, a people & object pager function, or a function of data transfer of a host device by grap. At least one of the above-mentioned functions may be supported for the first electronic device 100 independently or in association with the second electronic device 200.

For the biometric U-health function, at least on of pulse rate information, blood pressure information, oxygen saturation information, weight information, or body temperature information are required. The first device control module 160 may collect detection information using at least one sensor included in the first sensor module 110, and may support the biometric U-health function based on the collected detection information. The first communication module 160 may transfer, to the second electronic device 200, pulse rate information and blood pressure information collected while the biometric U-health function is performed. Thereafter, the second electronic device 200 may support the biometric U-health function based on the detection information received from the first electronic device 100.

The active oxygen measurement function is used to measure highly-oxidative unstable active oxygen in a body. Active oxygen may cause cancer, arteriosclerosis, diabetes, stroke, myocardial infarction, mesenchymal disease, nephritis, atopic dermatitis, and Parkinson's disease. The first device control module 160 may measure active oxygen using at least one sensor included in the first sensor module 110, and may compare the measured active oxygen and pre-collected information so as to notify a degree of danger of active oxygen and output guide information on related diseases. The active oxygen measurement function may be supported by execution of an integrated application 251 of the second electronic device 200. For example, the first device control module 160 may control the measurement of active oxygen and may provide a measured value of active oxygen to the second electronic device 200 so as to support the output of information on dangerousness of active oxygen-related diseases.

The blood pressure detection function is used to detect blood pressure. The first device control module 160 may measure a blood flow rate and a degree of vascular contraction or relaxation in a wrist using a combination of the EMG sensor 112 and the optical sensor 113, the EMG sensor 112 and the hall sensor 114, the EMG sensor 112 and the pressure sensor 115, or the EMG sensor 112 and the ECG sensor 117, so as to calculate blood pressure. A cardiovascular disease may include angina caused by a narrow coronary artery, contraction by spasm, or cholesterol deposition, a coronary artery disease, such as myocardial infarction or heat attack, hypertension in which highest blood pressure obtained when a heart contracts and lowest blood pressure obtained when a heart relaxes are higher than normal highest blood pressure and lowest blood pressure, malignant hypertension, cardiac insufficiency, brain hemorrhage causing cerebrovascular burst, and a stroke, such as cerebral infarction caused by blocked cerebral blood vessels. The first device control module 160 may perform blood pressure detection based on at least one piece of detection information collected by the sensors included in the first sensor module 110, and may output a result of the detection. In an embodiment of the present disclosure, information on the above-mentioned cardiovascular diseases or disorders may be output. Collected information related to blood pressure may be provided to the second electronic device 200. According to an embodiment of the present disclosure, the detection information collected by the first electronic device 100 may be provided to the second electronic device 200, and, accordingly, the blood pressure detection function may be provided by the second electronic device 200 as a function of the integrated application 251.

The low blood pressure notifying function may be used to output a notification if systolic blood pressure is about 90 mmHg or lower and diastolic blood pressure is, for example, about 60 mmHg or lower when blood pressure is measured by the first sensor module 110. In addition to the output of the blood pressure detection result, the first device control module 160 may perform the low blood pressure notifying function, and may output information on the symptoms of low blood pressure, such as fatigue, drowsiness, numbness in hands and feet, cold hands and feet, dizziness, faint, headache, and lethargy. Furthermore, the first device control module 160 may output information on the cause of low blood pressure, such as symptomatic or secondary low blood pressure (due to a cardiac disorder or endocrine disorder), essential low blood pressure (without particular symptom and clear cause), and orthostatic low blood pressure (due to a position change, such as sudden standing up after sitting or lying for a long time).

The first device control module 160 may provide a method of preventing low blood pressure. For example, the first device control module 160 may output information for instructing a user to lie sideways and take a rest when orthostatic low blood pressure occurs or information for instructing a user to exercise properly, sleep sufficiently and have a meal regularly when essential low blood pressure occurs. Furthermore, the first device control module 160 may output an instruction for making a request for searching for causes of symptomatic or secondary low blood pressure. The functions of collecting detection information related to blood pressure and outputting information on the symptoms and preventing method may be performed through the second electronic device 200. The first device control module 160 may collect the detection information related to blood pressure and may transfer the collected information to the second electronic device 200. The second electronic device 200 may support the low blood pressure notifying function based on the collected detection information related to blood pressure. The first device control module 160 may detect brain hemorrhage/myocardial infarction predicting signals, and may support a warning function depending on the detected signals.

The first device control module 160 may support a health maintaining function and a linking function installed in at least one of the first electronic device 100 and the second electronic device 200. The first device control module 160 may monitor a health status of a user. The first device control module 160 may continuously monitor biological signals so as to support continuous exercise management. For example, the first device control module 160 may measure a heart rate, a breathing rate, and an amount of exercise, and may support output of information for customized exercise management based on a result of the measurement. Furthermore, the first device control module 160 may store personal physical information indicating side effects of excessive or immoderate exercise for the purpose of obesity care, physical shape maintenance and health maintenance, and may read biological signals, such as a heart rate, caloric value and the number of operations to induce a user to exercise properly, so as to improve the effect of exercise. The first device control module 160 may measure biological signals of a heart related to the EMG sensor, and may support calculation of the number of operations and caloric value. Here, the first device control module 160 may display information for an operating sound state, may store a current state according to a heart rate, and may perform a function of notifying through a buzzer sound, e.g., notifying through a buzzer sound at intervals of 10 seconds.

The first device control module 160 may support the blood vessel measurement function using the EMG sensor and the optical sensor. In an embodiment of the present disclosure, the first device control module 160 may measure vascular health, vascular type, vascular age, and blood circulation. The first device control module 160 may measure Heart Rate Variability (HRV). The HRV represents a small variation between one cardiac cycle and a next cardiac cycle. A healthy person has a large and complex variation of a heart rate, but the complexity of a heart rate is remarkably reduced when the person suffers from a disease or is stressed. A normal heart rate is very irregular in an equilibrium state, due to an influence of an Autonomic Nervous System (ANS) affected by an environment in or outside a body.

A degree of the vascular health may be detected by simply diagnosing a degree of vascular aging and a degree of blood circulation. A vascular health index is affected by a vascular health type, but is closely related to life habits related to blood circulation. The first device control module 160 may provide information for changing life habits based on the vascular health index.

The first device control module 160 may support a pulse stress measurement function. A pulse variation graph shows a variation of heart heat. Here, a larger variation may represent stronger health. The first device control module 160 may determine that the pulse variation is normal if the pulse variation of an adult is, for example, about 30-40 on average and may determine that an adult user is under chronic stress if the pulse variation is about 20 or lower. The first device control module 160 may output information corresponding to a state where there is almost no stress if the pulse variation is about 25 or lower and may output information corresponding to a state where there is temporary stress if the pulse variation is about 25-35 inclusive, through the pulse stress measurement function. The first device control module 160 may output information corresponding to a state of initial stress if the pulse variation is about 35-45 inclusive, may output information corresponding to a period in which temporary stress is repeated and stress resistance is weakened if the pulse variation is about 45-60 inclusive, and may output information corresponding to a state of progressing to chronic stress.

The first device control module 160 may support the medicine taking assistant function. The first electronic device 100 may increase a medicine taking acceptance ratio of people with chronic diseases who need to continuously take medicines, so as to reduce medical expenses. An aged person may forget that he or she has taken a medicine and may take an overdose of a medicine, and thus, an emergency situation may occur. To prevent such a situation, the first device control module 160 may support the medicine taking assistant function so as to notify a time for taking a medicine and notify an operating center or medical team of the fact that a medicine has been taken. The first device control module 160 may establish a communication channel to the second electronic device 200 or other external devices, such as a digital photo frame and a smart TV, in order to support medicine taking of a person with a chronic disease or an aged person. According to various embodiments of the present disclosure, the first device control module 160 may register a medicine taking schedule and may provide, in the form of a voice and screen, guidance for medicine taking at a medicine taking time if medicines are accommodated in a medicine box. The medicine taking assistant function may be performed through the second electronic device 200 or may be performed by execution of an integrated application of the second electronic device 200. Accordingly, a user may support exercise management, diet management and health measurement management of a person with a chronic disease or an aged person by using a touch screen of the second electronic device 200. The first device control module 160 may perform the medicine taking assistant function through various external devices, such as a digital photo frame, a wireless mobile terminal, a smart TV, and the like. Furthermore, the first device control module 160 may support a function of detecting a text message regarding an untaken medicine. Furthermore, the first device control module 160 may support a management function for medicine administration by hospitals, guardians or institutions through information update of an integrated website. The first device control module 160 may support a medicine taking information view function, a function of reminding of taking a medicine through an LED display, a liquid crystal display or a voice, and a diet/exercise/health measurement management function using the second electronic device 200.

The first device control module 160 may support the fall detection function. The fall detection function may be used to detect the occurrence of a fall and send a location-based distress signal so that a fall accident of an aged person is recognized rapidly. For example, the first device control module 160 may perform a control operation so that a specific message is sent to an operating center or a family together with location information when a fall accident and emergency situation occur, by using the detection information collected by the acceleration sensor 116. According to an embodiment of the present disclosure, the first device control module 160 may collect detection information related to fall detection using a tri-axial acceleration sensor and may make a correct determination about a fall detection situation through multi-stage filtering. When it is determined that a fall has occurred, the first device control module 160 may support a function of calling a communication device arranged in a specific emergency facility, a function of notifying a situation through the second electronic device 200, and a function of tracing a location of a patient having an injury from a fall in association with the second electronic device 200.

In connection with the detection of a fall accident, the first device control module 160 may perform action detection and heart rate measurement. Information about the action detection may be obtained using the tri-axial acceleration sensor 116. In connection with the action detection, the occurrence of action may be determined based on a detection variation of the acceleration sensor. When a fall occurs, rapid and large motions are repeated for a short time. Although a rapid motion may also occur in a situation, such as shaking hands, a variation of a signal wave caused by such a motion is not large, and thus, such a motion may be distinguished from a fall. Therefore, the first device control module 160 may distinguish an intentional rapid hand motion from a momentary motion, such as a fall. For example, the first device control module 160 may differentiate a handshaking state, an arm tremor state, a hand raising state, a walk state, and a fall state through modeling of acceleration detection information.

The first device control module 160 may support the living-alone senior monitoring function. Based on this function, the first electronic device 100 may support prevention of lonely deaths of living-alone seniors. For example, the first electronic device 100 may be kept worn on a wrist of a living-alone senior. The first device control module 160 may periodically monitor a pattern and heart rate of a user so as to support an operation for treating the case where detection information is rapidly changed. For example, the first device control module 160 may automatically perform a living-alone senior management function in association with the second electronic device 200, and may request the second electronic device 200 to call a specific relief facility.

The first device control module 160 may support the snoring prevention function. The first device control module 160 may measure an oxygen saturation of a wrist using the first sensor module 110, and may detect a pulse flow based on the measured oxygen saturation. The first device control module 160 may perform a control operation so that certain stimulation is provided in order to stop snoring when a user snores. When a person sleeps, tense muscles are relaxed. A muscle of a neck of a snoring person is excessively relaxed, and thus, the neck muscle is easily trembled by a light breath, causing snoring. The first device control module 160 may perform a control operation so that a muscle of a throat is constantly tensed through a muscle of a wrist. Alternatively, the first device control module 160 may provide a certain vibration using the vibration module 180, so as to control a muscle motion of a wrist.

The first device control module 160 may support the recipe providing function. The first device control module 160 may collect biological signals collected by at least one of the first sensor module 110 and the second sensor module 120 for a certain period of time, and may transfer the collected biological signals to the second electronic device 200 after a lapse of a certain time. For example, the first device control module 160 may transfer various signals about a heartbeat, blood pressure, respiration, oxygen saturation and the like to the second electronic device 200. The second electronic device 200 may detect an abnormal symptom and health state based on the collected biological signals, and may collect diet information corresponding to the health condition. The collected diet information may output through a display module of the first electronic device 100 or an external electronic device, such as a communicable smart TV.

The first device control module 160 may support the safe driving assistant or drowsy driving prevention function in combination with a U-health care system. The first device control module 160 may perform real-time automatic monitoring even when a user is in a dynamic state, such as driving a vehicle, so as to overcome the limitation of a health management system in which health management is performed in a static state in homes or hospitals. The first device control module 160 may detect a state of a driver in association with a navigation system and may collect biological signals of a current state (pulse, body temperature, and the like) of the driver, and then may provide driving assistance information based on the biological signals. For example, the first device control module 160 may notify that a pulse is rapid or a body temperature is high in a voice form so as to warn a user or provide instructions on a next action to the user. The first device control module 160 may output warning information through an electronic device arranged in a vehicle, or may output biological information in association with the second electronic device 200. The first device control module 160 may determine whether a user drives while drowsy based on various pieces of information related to biological signals, for example, detection information, such as pulse measurement information or atmosphere environment measurement information, and may output a warning sound or warning vibration according to a result of the determination.

The first device control module 160 may support the function of detecting an abnormal environment state using the olfactory sensor 123. The first device control module 160 may detect a rotten material or detect an abnormal environment state using the olfactory sensor 123. The olfactory sensor 123 may detect and quantify specific gas in the air.

The first device control module 160 may support the function of measuring sweat, moisture or secretion of a body using the taste sensor 121. The taste sensor 121 for measuring sweat may noninvasively perform information detection. The first device control module 160 may measure various harmful germs and smells generated from sweat, moisture or secretion of a body.

The first device control module 160 may support the business card recognition function (PAN). A human body has various characteristics of response to an electrical signal depending on a frequency change. A carrier frequency used for communication may be changed according to characteristics of a human body medium. The first device control module 160 may detect and set a carrier frequency most suitable to human body communication. The first device control module 160 may transmit a signal with a carrier frequency of up to about 50 MHz through a human body in order to measure a frequency characteristic, and may measure a radiation effect by detecting a transmitted attenuation amount. When the carrier frequency for human body communication is detected, the first device control module 160 may transmit business card information through contact between users. The business card information may be pre-stored in a memory of the first electronic device 100. The first device control module 160 may collect price information on a specific object through human body communication and may output the price information.

The first device control module 160 may support the bone conduction phone function. In associated with bone conduction phone function, the first device control module 160 may generate a vibration corresponding to an audio signal through the vibration module 180.

The first device control module 160 may support the anti-static function. The first electronic device 100 may be worn on a wrist to continuously absorb static electricity of a body through the wrist and discharge the static electricity into the air.

The first device control module 160 may provide a context-aware-based U-health environment information service. In associated with the context-aware-based U-health environment information service, the first device control module 160 may analyze and process internal information corresponding to biological signals and external information obtained by various sensors, and may output service information provided by the second electronic device 200 or the server device 400.

The first device control module 160 may support the biometric based emotion recognition service function. The first device control module 160 may detect a change of an emotion of a person due to a pulse wave that is a type of response of an autonomic nervous system (ANS), a human body temperature and skin resistance, an external temperature, external humidity and external noise. For example, the first device control module 160 may collect biological signals and external signals that affect the emotional change by using at least one of the sensors included in the first and second sensor modules 110 and 120. The first device control module 160 may provide the collected biological signals and external signals to the second electronic device 200 or server device 400, may receive emotion change information corresponding to the provided signals, and may output the received emotion change information. Such an emotion recognition service function may be applied to a new industry field of emotional convergence, such as an emotional convergence entertainment industry, an emotional convergence digital life industry, an emotional convergence education industry, an emotional convergence community industry, an emotional convergence commerce industry, and an emotional convergence Emotion On Demand (EOD) industry.

The first device control module 160 may support the Parkinson's disease diagnosis function. A Parkinson's disease, which is a type of a chronic degenerative disease of a nervous system, results from the death of dopamine nerve cells distributed in the substantia nigra, and has symptoms, such as shaking, rigidity, slowness of movement and unstable posture. The first device control module 160 may collect biological signal information corresponding to the four major symptoms of the Parkinson's disease. For example, the first device control module 160 may collect and analyze biological signals that enable analysis on the symptoms of shaking, rigidity, slowness of movement and unstable posture. In connection with the symptom of shaking, the first device control module 160 may detect rhythmic shaking from terminal joints, such as finger or wrist joints, or may detect shaking with a frequency of about 4-6 Hz. The first device control module 160 may determine whether the Parkinson's disease occurs based on accumulated information of biological signals. For example, the first device control module 160 may output warning information when legs, chin or tongue shakes on both sides of a body after a lapse of a certain period of time from the time of occurrence of shaking on one side of the body. The first device control module 160 may support a Parkinson's disease diagnosis mode, and may measure an angular motion by combining detection values of a gyro sensor, an acceleration sensor, and an acoustic vibration sensor.

The first device control module 160 may support the lie detection function. The lie detection function, which is a type of polygraph, is used to determine whether a statement of a suspect is true based on various responses of an ANS caused by subjective symptoms and mental changes. The first device control module 160 may measure respiration, blood pressure, pulse and a voltage change of electric resistance of a hand caused by an unstable mental state, and may determine whether a statement is the truth or a lie based on a result of the measurement. Here, the first device control module 160 obtains a pulse signal through SPO₂, and measures the voltage change with respect to the electric resistance of a hand. The first device control module 160 may analyze a pulse of a user and electric resistance of a hand based on the fact that a pulse rate becomes higher and the electric resistance is decreased due to sweat from a hand when a person tells a lie.

The first device control module 160 may support the function of recognizing tennis swing using an acceleration sensor. The first electronic device 100 may be worn on a wrist of a user to recognize motions of five types of tennis swing. Here, the first device control module 160 may analyze a characteristic of an acceleration sensor value depending on a wrist motion, and may recognize a tennis motion based on Discrete Wavelet Transform (DWT). The tennis swing may include, for example, forehand stroke, backhand stroke, service, forehand volley, and backhand volley.

The first device control module 160 may analyze a form of a discrete signal obtained from an acceleration sensor of each axis according to the motions of forehand stroke, backhand stroke, forehand volley, backhand volley and service. When a sensor value of each axis is analyzed in a motion operation, the first device control module 160 may determine how the position and direction of a wrist change while swing is performed. For example, when backswing, which is a motion of rotating a body in an opposite direction to a ball hitting direction, is performed, the first device control module 160 may obtain a sensor value corresponding to an end motion of the backswing so as to analyze a tennis swing motion.

The first device control module 160 may support the fieldwork function. The first device control module 160 may record peripheral environment information when a fieldwork, such as wild animal observation, is performed. The first device control module 160 may automatically input information, such as a current location and time to an electric map. In order to collect current location information, the first electronic device 100 may include an additional location information collection module or may use a location information collection module of the second electronic device 200.

The first device control module 160 may support the home network function. The first device control module 160 may obtain information on various situations, such as a user situation, a physical environment situation, a computing system situation, a history of interaction between a user and a computer, and other non-classified situations through various physical devices, such as a sensor, a network, an actuator and a presentation device, and may provide an active service corresponding to the obtained information. For example, the first device control module 160 may assign a unique right to a user by using information obtained in an active context-aware-based home network environment, and may provide a service suitable for each user having a right. For example, the first device control module 160 may differentiate actions of turning on a TV and gas stove by parents and children. For example, the first device control module 160 may assign a right to turn on a TV and gas stove to parents, but may not allow children to access a TV and gas stove. The first device control module 160 may support the education and safety of children by using a context-aware function under a home network environment.

The first device control module 160 may provide the U-themepark service function for detecting a location of a user and collecting and providing information on concerts and rides adjacent to the detected location. The first device control module 160 support the ComMotion function for transmitting a location-based memorandum configured to be transmitted when a specific electronic device approaches a specific location. When the specific electronic device approaches the location, the first device control module 160 may automatically transmit the memorandum. When the first device control module 160 receives the memorandum, the first device control module 160 may convert the memorandum into a voice and may output the voice.

The first device control module 160 may provide the shopping assistant function for providing services, such as a shopping guide, product detailed information search, product location search, and search for products on a sale, according to a location of a user. The first device control module 160 may provide the cyber-guide function for providing services, such as background information for navigation and a location, and automatic writing of a travel diary. The first device control module 160 may provide the people & object pager function for displaying information, such as a name of a presenter and a title of a presentation when the presenter enters a presentation place and for searching for available A/V devices to automatically record the presentation.

The first device control module 160 may recognize a location of a visitor or an object to route a message to a most adjacent person. The first device control module 160 may provide relevant information based on fields of interest of a user according to a varying location of the user. A current location of the user may determine a time for providing information. A location change due to a motion may be an input for providing information implicitly. Information provided in a virtual space and a real space may be filtered according to an interest of the user. For example, when a user wearing the first electronic device 100 arrives at a place A, the first electronic device 100 may receive information from an information generator of a real space, but the first electronic device 100 may perform a control operation so that the information is filtered out and is not output when the place A does not relate to an interest of the user. The interest (for example, value of inferences based on an internal situation of the electronic device) of the user may be continuously modeled based on an action, such as an information search in a virtual information space, such as a web environment. An external situation of the user may be recognized through a Global Positioning System (GPS) or a sensor for recognizing a location.

The external situation of the user which determines a time for providing information may be time information in the case of providing information related to a schedule. When a certain time arrives, the first device control module 160 may automatically perform a music playing function at the time. For example, provided music may be variously changed based on an interest of a user. The first device control module 160 may collect history information on device operation, and may extract a device operation pattern based on the collected history information. According to the extracted device operation pattern, the first device control module 160 may support automatic performance of a specific function, such as a music playing function or a specific service information collection function.

When the first electronic device 100 is located at a specific place and receives information provided from the periphery of the place, e.g., digital graffiti content, the first device control module 160 may filter and output the graffiti content according to a human relationship of a user. Here, the human relationship of the user may be modeled and provided based on an electronic mail address book of the user, a chatting history in a virtual space, and a messenger service. The first device control module 160 may automatically adjust the scope of the human relationship so that an amount of received information is not greater than a certain amount.

The first device control module 160 may support the function of transferring data of a host device to anther device by grap. The first device control module 160 may maintain the first electronic device 100 in a data reception standby state. An optical sensor of the second electronic device 200 may detect the approach of the first electronic device 100. When a gesture for selecting a specific file or data of the second electronic device 200 occurs, the first electronic device 100 worn on a wrist of a user may recognize the gesture of the user and may transfer the recognized gesture to the second electronic device 200 so as to request transmission of specific data.

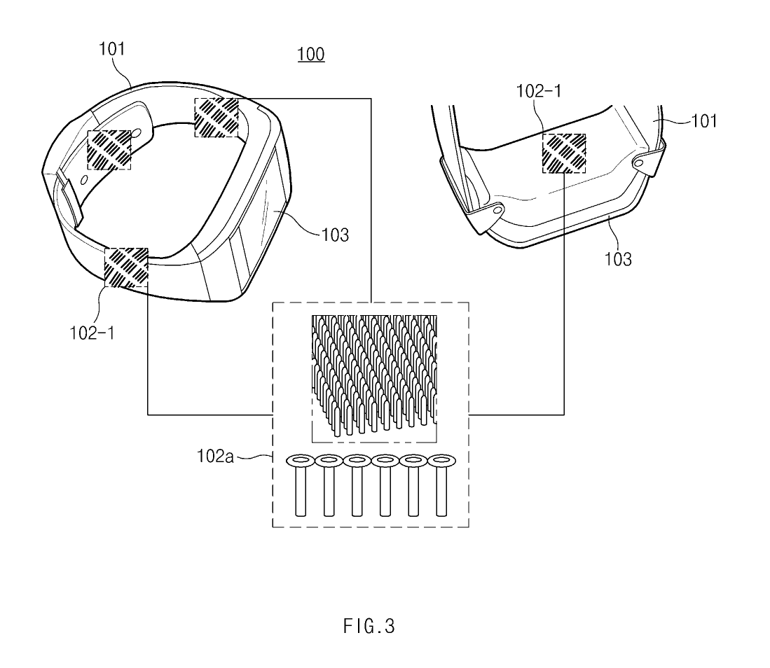

FIG. 3 is a schematic diagram illustrating an outward appearance of a first electronic device according to an embodiment of the present disclosure.

Referring to FIG. 3, the first electronic device 100 may have such a structure as to tightly contact with a wrist. For example, the first electronic device 100 may include a display body 103, a connection body connected to both sides of the display body 103 so as to tightly contact with a side portion of a wrist, and a first-type sensor module 102-1 arranged at one side of the display body 103 and connection body 101. The first-type sensor module 102-1 may include at least one of the above-mentioned first sensor module 110 and second sensor module 120.

The display body 103 may include the first display module 140, the first audio module 170, the first communication module 190, and the vibration module 180. Coupling structures to be coupled with the connection body 101 may be arranged at both sides of the display body 103. In an embodiment of the present disclosure, the coupling structures may include various structures, such as a hook structure or a screw structure.

The connection body 101 may include connection parts respectively connected to both sides of the display body 103, and a fastening part for connecting the connection parts at the ends thereof so that the connection parts tightly contact with a wrist. Although the fastening part has a folded shape in the drawings, the structure of the first electronic device 100 is not limited thereto. For example, the fastening part may include at least one hole arranged at one connection part, and a buckle structure that may be inserted to the hole.

The first-type sensor module 102-1 may be arranged at least one side of the connection body 101, e.g., edges or the vicinity of a boundary of the display body 103 and connection parts facing a wrist, a bottom surface of the display body 103, and the fastening part. The first-type sensor module 102-1 arranged at the vicinity of the boundary of the connection parts may tightly contact with side portions of a wrist so as to detect a radial artery of the wrist and changes of muscles related to the radial artery. The first-type sensor module 102-1 arranged at the bottom surface of the display body 103 may tightly contact with an upper end portion of a wrist so as to detect a change of a motion of extensor digitorum. The first-type sensor module 102-1 arranged at the fastening part may detect a hand artery and changes of muscles related to the hand artery. As illustrated in FIG. 3, the first-type sensor module 102-1 may have a shape in which a plurality of micro cilia 102a are uniformly arranged. The first-type sensor module 102-1 having such a structure may bring about a bonding effect without causing stickiness on a skin. By partially attaching a dry adherer to a portion of a skin having a largest surface area, a watch may be prevented from moving on a wrist as if the watch were bonded to a skin. In order to apply a dry adhesive patch to a surface of a human body which is rough and secretes fat and moisture and constantly moves, a dry adhesive structure obtained by mixing a cilium structure of a gecko lizard to which the van der Waals' force is applied with a sucker structure of an octopus is used. An octopus may freely move in the water by using a distinctive sucker structure. By using such a characteristic, the limitation of the secretion of a human body skin may be overcome. Furthermore, the first-type sensor module 102-1 may have such a structure as to minimize a noise generated during a detection process.

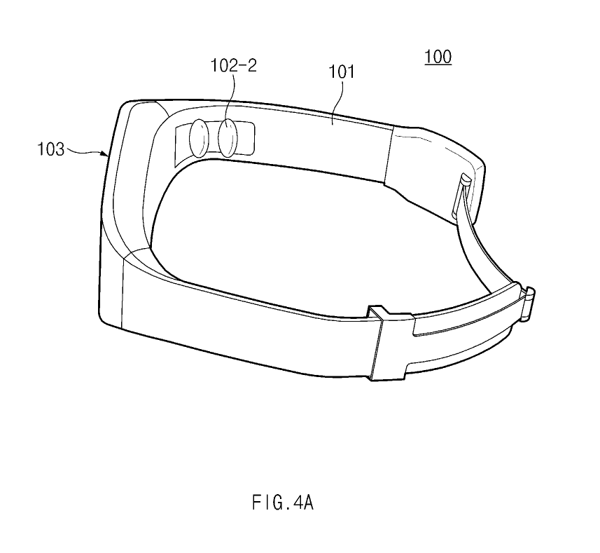

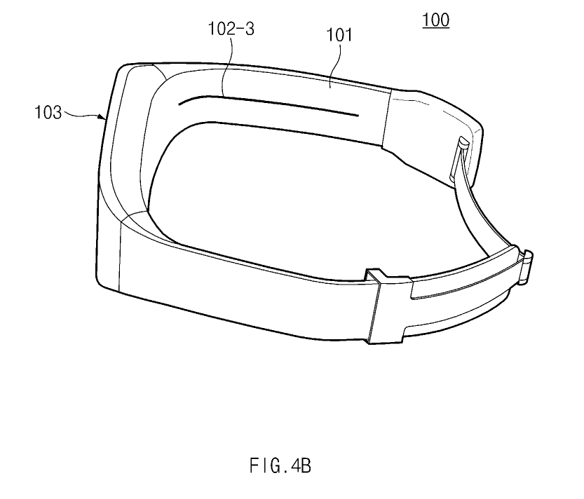

FIG. 4A is a schematic diagram illustrating a first electronic device according to an embodiment of the present disclosure.

Referring to FIG. 4A, the first electronic device may include the display body 103, the connection body 101, and a second-type sensor module 102-2 arranged at one side of the connection body 101. The display body 103 and the connection body 101 may have shapes similar to the shapes described above with reference to FIG. 3.

The second-type sensor module 102-2 may be arranged at one side of the connection body 101. For example, as illustrated in FIG. 4A, the second-type sensor module 102-2 may be arranged at one side of a connection region of the connection body 101 which is connected to the display body 103. The second-type sensor module 102-2 may include at least one protrusion so as to tightly contact with a wrist. The protrusion may protrude from a surface of the connection body 101. When a fastening operation of the connection body 101 is performed while the first electronic device 100 is worn on a wrist, the protrusion of the second-type sensor module 102-2 may contact with a certain portion of the wrist, e.g., a side portion of the wrist. The protrusion of the second-type sensor module 102-2 may be provided in a plurality in order to support the first electronic device 100 so that the first electronic device 100 does not rotate around a wrist.

FIG. 4B is a schematic diagram illustrating a first electronic device according to an embodiment of the present disclosure.

Referring to FIG. 4B, the first electronic device may include the display body 103, the connection body 101, and a third-type sensor module 102-3 arranged at one side of the connection body 101. The display body 103 and the connection body 101 may have shapes similar to the shapes described above with reference to FIG. 3. When a fastening operation of the connection body 101 is performed while the first electronic device 100 is worn on a wrist, a shape memory alloy material arranged at the third-type sensor module 102-3 may contact with a certain portion of the wrist, e.g., a side portion of the wrist, so as to secure the first electronic device 100 to the wrist. In connection with this operation, a shape may be memorized in a certain material in an environment where a certain time and temperature are maintained in order to provide the first electronic device 100. The first electronic device 100 provided in this manner may be restored to an original shape even though the first electronic device 100 is bent or twisted by external force.

According to various embodiments of the present disclosure, the first electronic device 100 may include the first sensor module 110 including the EMG sensor 112 for detecting a muscle state of a wrist and an auxiliary sensor for detecting biological signals about a state of biological tissue of the wrist, the first display module 140 for outputting information related to the biological signals collected by the first sensor module 110, and the first device control module 160 for controlling the first sensor module 110 and controlling the output of the information.

According to various embodiments of the present disclosure, the first sensor module 110 may detect a state of at least one of extensor digitorum and extensor digiti minimi of a wrist.

According to various embodiments of the present disclosure, the first sensor module 110 may detect a state of at least one of a radial artery and an ulnar artery of a wrist.

According to various embodiments of the present disclosure, the auxiliary sensor may include the acoustic vibration sensor 111, optical sensor 113, pressure sensor 115, ECG sensor 117 and hall sensor 114 for detecting a state of vessels or muscles of a wrist, and the acceleration sensor 116 for collecting signals generated due to a motion of the wrist.

According to various embodiments of the present disclosure, the first electronic device 100 may further include the second sensor module 120 including at least one of the taste sensor 121 for detecting a secretion of a user and the olfactory sensor 123 for collecting smell information on a peripheral environment of the user.

According to various embodiments of the present disclosure, the first electronic device 100 may include the display body 103 including the first display module 140 and first device control module 160, and the connection body 101 connected to both sides of the display body 103, and the first sensor module 110 may include at least one sensor arranged at a surface on an edge of the connection body 101 connected to the display body 103, the surface facing a wrist.

According to various embodiments of the present disclosure, the first electronic device 100 may further include the first communication module 190 for transmitting the collected biological signals to the second electronic device 200.

According to various embodiments of the present disclosure, the first communication module 190 may receive, from the second electronic device 200, service information related to the biological signals.

According to various embodiments of the present disclosure, the first display module 140 may be an electronic device that outputs at least one of information on an ECG, heart rate, blood oxygen saturation or blood pressure among the biological signals and information on a stress index related to a pulse among the biological signals.

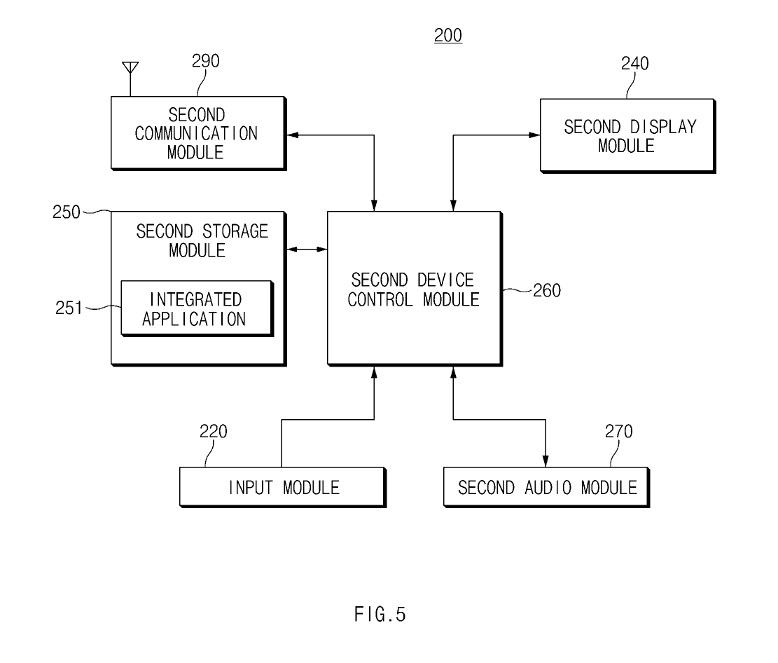

FIG. 5 is a block diagram illustrating a second electronic device according to an embodiment of the present disclosure.

Referring to FIG. 5, the second electronic device 200 may include an input module 220, a second display module 240, a second storage module 250, a second audio module 270, a second communication module 290, and a second device control module 260.

The second electronic device 200 having the above-mentioned structure may establish a communication channel to the first electronic device 100 to receive biological signals from the first electronic device 100. The second electronic device 200 may perform various functions related to the received biological signals and may collect biological signal-related service information. In connection with the collection of the biological signal-related service information, the second electronic device 200 may establish a communication channel to the server device 400. The second electronic device 200 may output the biological signal-related service information received from the server device 400 or may transfer the biological signal-related service information to the first electronic device 100.

The input module 220 may generate various input signals related to operation of the second electronic device 200. For example, the input module 220 may include a key button, a touch key, a touch pen, a pen panel, a gesture input, and a microphone module MIC for recognizing a voice. Furthermore, in the case where the second display module 240 is provided as a touch screen, the input module 220 may include the second display module 240 as an input unit. The input module 220 may generate an input signal for requesting a communication channel to the first electronic device 100 and an input signal for requesting collection of biological signal-related service information. According to an embodiment of the present disclosure, the input module 220 may generate an input signal for requesting execution of the integrated application 251 based on the biological signals provided by the first electronic device 100 and an input signal for requesting termination of the integrated application 251. The generated input signals may be transferred to the second device control module 260 so as to be operated as a command set for controlling a corresponding function, such as activation or termination of the integrated application 251.

The second display module 240 may output various screens related to operation of the second electronic device 200. For example, the second display module 240 may output a standby screen, a menu screen, a screen on which at least one icon is arranged, and a lock screen of the second electronic device 200. According to an embodiment of the present disclosure, the second display module 240 may output a screen on which an icon or menu item related to activation of the integrated application 251 is arranged. When the integrated application 251 is selected, the second display module 240 may output a screen for establishing a communication channel to the first electronic device 100 and a screen related to biological signals provided by the first electronic device 100. The second display module 240 may output a screen for performing access to the biological signal-related server device 400 and a screen including the biological signal-related service information provided by the server device 400.

The second storage module 250 may store various programs and data related to operation of the second electronic device 200. For example, the second storage module 250 may store an operating system. The second storage module 250 may store the integrated application 251. The integrated application 251 may perform a specific function or may provide the biological signal-related service information in connection with the biological signals provided by the first electronic device 100. The type or function of the integrated application 251 will be further described in connection with a service function according to processor operation of the second device control module 260.

The second audio module 270 may support an audio-related function of the second electronic device 200. For example, the second audio module 270 may output an audio of music played by the second electronic device 200 and an audio signal received from other electronic device. Furthermore, the second audio module 270 may collect or output a voice signal when a call function of the second electronic device 200 is supported. According to an embodiment of the present disclosure, the second audio module 270 may output an audio signal related to reception of biological signals from the first electronic device 100. Furthermore, the second audio module 270 may output an audio signal related to processing of biological signals and collection of biological signal-related service information. Here, the audio signal may be a guide sound or a specific effect sound. The output of the guide sound or effect sound of the second audio module 270 may be omitted according to control by a user or a design method.

The second communication module 290 may perform a communication function of the second electronic device 200. In the case where the second electronic device 200 supports a mobile communication function, the second communication module 290 may include a mobile communication module. The second communication module 290 may include a communication module related to a broadcast reception function and a communication module related to a short-range communication function. According to an embodiment of the present disclosure, the second communication module 290 may establish a communication channel to the first communication module 190 arranged in the first electronic device 100. The second communication module 290 may receive biological signals from the first electronic device 100. The second communication module 290 may transfer, to the first electronic device 100, various pieces of information generated due to execution of the biological signal-related integrated application 251. Furthermore, the second communication module 290 may establish a communication channel to the server device 400 and may receive the biological signal-related service information from the server device 400. The second communication module 290 may transmit the received biological signal-related service information to the first electronic device 100.

The second communication module 290 may include a communication module capable of establishing a communication channel to the server device 400 through the communication network 300 and a communication module capable of establishing a communication channel to the first electronic device 100. The second communication module 290 may be a communication module that supports one communication mode, or may include a plurality of communication modules that support a plurality of communication modes. For example, the second communication module 290 may include a plurality of communication modules so as to establish a short-range communication channel to the first electronic device 100 and a long-range communication channel to the server device 400.

The second device control module 260 may perform signal processing and transferring and data processing and transferring related to operation of the second electronic device 200. According to an embodiment of the present disclosure, the integrated application 251 of the second device control module 260 may perform various processes related to performance of a biological signal-related function or output of biological signal-related service information. The integrated application 251 may support at least one of various service functions related to biological signals. Alternatively, the integrated application 251 may include a plurality of applications related to biological signals. The second device control module 260 may include at least one processor in association with a plurality of applications.

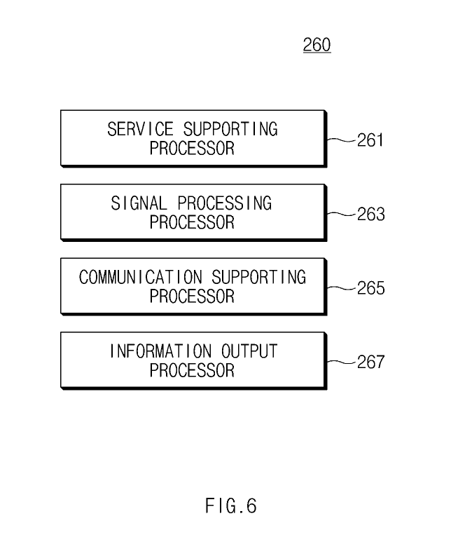

FIG. 6 is a diagram illustrating a second device control module according to an embodiment of the present disclosure.

Referring to FIG. 6, the second device control module 260 may include a service supporting processor 261, a signal processing processor 263, a communication supporting processor 265, and an information output processor 267.

The service supporting processor 261 may support execution of the integrated application 251 and processing of various data generated due to the execution of the integrated application 251. For example, the service supporting processor 261 may support output of an icon or menu item for activating the integrated application 251. When the activation of the integrated application 251 is requested, the service supporting processor 261 may call the communication supporting 265 so as to establish a communication channel to the first electronic device 100.

When biological signals are received from the first electronic device 100, the communication supporting processor 265 may transfer the biological signals to the signal processing processor 263. When the biological signals are received, the communication supporting processor 265 may transfer the biological signals to the specific server device 400 and may receive the biological signal-related service information from the server device 400. The received biological signal-related service information may be transferred to the information output processor 267.

The signal processing processor 263 may process the biological signals received from the first electronic device 100 while the integrated application 251 is executed. For example, the signal processing processor 263 may determine what type of a hand motion has been performed based on EMG detection information and optical detection information included in the received biological signals.

The information output processor 267 may output, to the second display module 240, the biological signals received by the communication supporting processor 265 from the first electronic device 100 and the biological signal-related service information received from the server device 400. Furthermore, the information output processor 267 may output relevant information through the second audio module 270.

The second device control module 260 may support various service functions related to biological signals by using the above-mentioned processors 261, 263, 265 and 267. For example, the second device control module 260 may support a biometric U-health support function, an active oxygen information providing function, a blood pressure information providing function, a low blood pressure notifying function, a function of notifying signals of a brain hemorrhage or myocardial infarction, a blood vessel measurement information providing function, a pulse wave-based stress information providing function, a medicine taking assistant function, a fall detection function, an action detection function, a heart rate information providing function, a living-alone senior monitoring function, a snoring prevention function, a recipe providing function, a safe driving assistant or drowsy driving prevention function, a function of providing information on an abnormal environment state based on biological signals collected by an olfactory sensor, a function of providing information on sweat, moisture or secretion based on biological signals collected by a taste sensor, a business card recognition function (PAN), a bone conduction phone support function, a biometric based emotion recognition service function, a Parkinson's disease information providing function, a lie detection information providing function, a function of supporting recognition of tennis swing using an acceleration sensor, a fieldwork support function, a home network support function, a U-themepark service support function, a ComMotion support function, a shopping assistant support function, a cyber-guide support function, a conference assistant support function, a people & object pager support function, and a function of supporting data transfer of a host device by grap. As described above, at least one of the above-mentioned functions may be independently supported in the first electronic device 100 according to control by the first device control module 160.

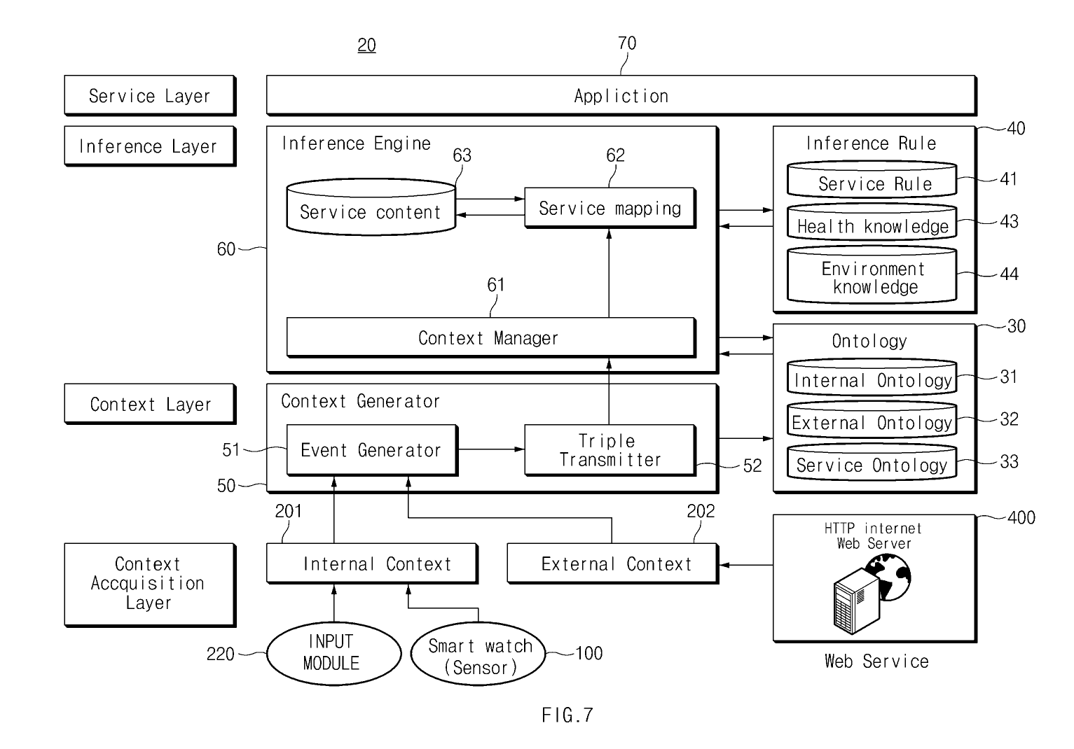

FIG. 7 is a diagram illustrating a function operation layer of a second device control module according to an embodiment of the present disclosure.

Referring to FIG. 7, a function operation layer 20 may include a context acquisition layer, a context layer 50, an inference layer 60, and a service layer 70, and may include an inference rule module 40 and an ontology module 30.

The context acquisition layer may include an internal context module 201 that receives information input by a user through the input module 220 or receives biological signals transferred by the first electronic device 100 and an external context module 202 that receives service information provided by the server device 400.

The context layer 50 may include an event generator 51 and a triple transmitter 52. The event generator 51 receives information from the internal context module 201 and the external context module 202, generates an event for the received information, and transfers the event to the triple transmitter 52. The triple transmitter 52 may transfer the generated event to a context manager 61. Here, the triple transmitter 52 may transfer the generated event to the ontology.

The inference layer 60 may include the context manager 61, a service mapping unit 62, and a service content unit 63. The context manager 61 transfers, to the service mapping unit 62, the event received from the triple transmitter 52. Here, the context manager 61 may determine an ontology corresponding to the event. The service mapping unit 62 maps ontology information corresponding to the event transferred by the context manager 61 to service content stored in the service content unit 63. During a service mapping process, the service mapping unit 62 may use a service rule 41 included in the inference rule module 40, health knowledge 43, and environment knowledge 44.

The service layer 70 may output the service content information mapped by the service mapping unit 62. The ontology module 30 may include an internal ontology module 31, an external ontology module 32, and a service ontology module 33. The ontology module 30 is used to generalize low-level information and high-level information and define a relation between pieces of information. The ontology module 30 performs inference of context data according to a service and supports information extension according to the inference.

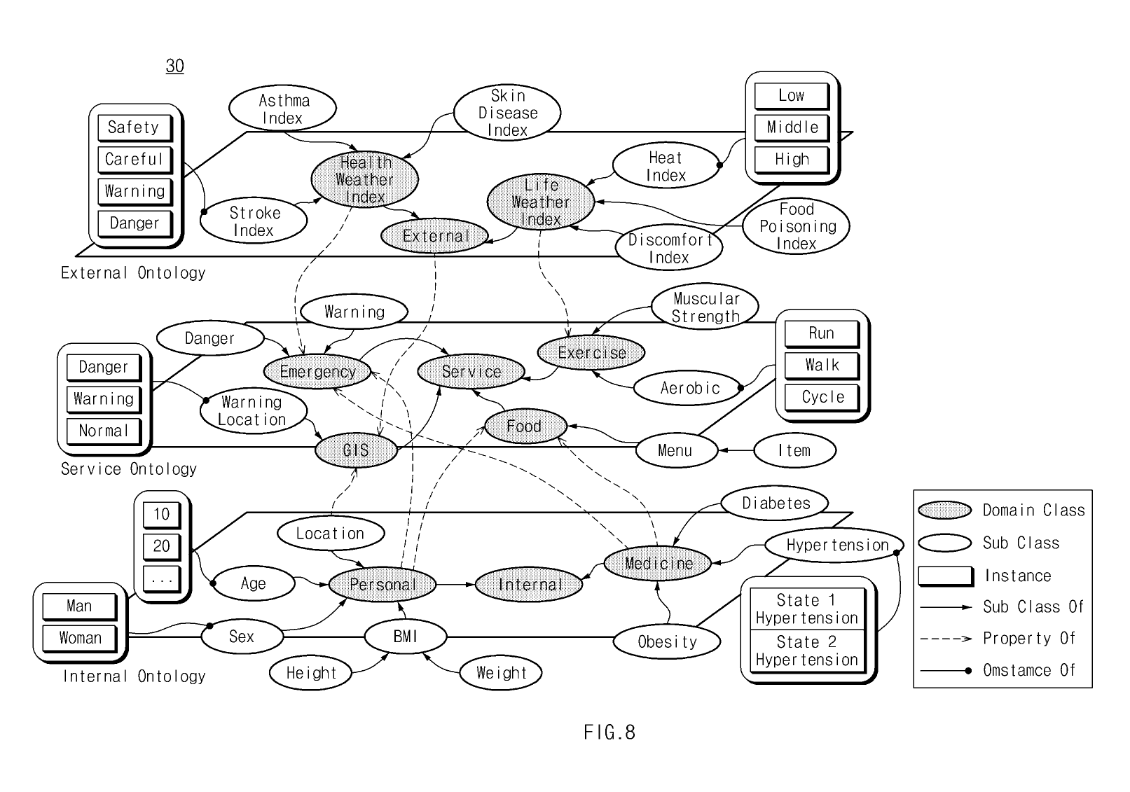

FIG. 8 is a diagram illustrating an ontology module according to an embodiment of the present disclosure.

Referring to FIG. 8, the ontology module 30 may include the internal ontology module 31, the service ontology module 33, and the external ontology module 32.

The internal ontology module 31 has a form for inferring a state of a user, and a high class may include a personal class and a medicine class. The personal class, which defines basic information of the user, has information about sex, age, Body Mass Index (BMI), location, height and weight. The medicine class has information about hypertension, diabetes, obesity and asthma. The second device control module 260 may infer whether the user has a disease and five types of health level.

The external ontology module 32 may include a health weather index class and a life weather index class. The health weather index class has information about a city high-temperature index, a stroke index, an asthma index, a pollen concentration index, a skin disease index, and a pulmonary disease index, and may infer a degree of danger with respect to each index and location.

The health weather index is used to represent a degree of effect of a part or group of weather elements on a life by using an index. For example, an index of a scientific quantitative indication method is used for an unscientific qualitative indication method.

The health weather index of a weather center is used to provide the asthma index, stroke index, skin disease index, pulmonary disease index, pollen concentration index and city high-temperature index.

The stroke index is obtained by indexing a degree of possibility of occurrence of a stroke according to weather conditions, such as a temperature, atmospheric pressure and humidity and non-weather factors, such as seasonal factors. A stroke affects and indirectly damages a cardiovascular system and a cerebrovascular system. When an external temperature decreases, a blood vessel contracts, blood pressure increases, and the viscosity of blood in a body increases, and thus, friction of blood increases, which slows a blood flow, increases possibility of generation of a blood clot and causes the blockage or burst of a blood vessel, thereby causing hypertension and stroke.

The asthma index is obtained by indexing a degree of possibility of occurrence of asthma according to weather conditions, such as a temperature, atmospheric pressure and humidity and non-weather factors, such as seasonal factors. A high temperature increases the ozone concentration and thus affects an allergic respiratory disease, such as asthma. Ozone is a secondary pollutant generated due to reaction between an air pollutant, such as a nitrogen oxide and ultraviolet light, and causes serious harm to an asthma patient by stimulating a respiratory organ. The asthma index may be calculated by synthesizing indices of pollen, yellow dust, and cold (temperature/humidity). The asthma index may have a numerical value between 0 and 100.

The skin disease index is obtained by indexing a degree of possibility of occurrence of a skin disease according to weather conditions, such as a temperature, atmospheric pressure and humidity and non-weather factors, such as seasonal factors. The pulmonary disease index is obtained by indexing a degree of possibility of occurrence of a pulmonary disease according to weather conditions, such as a temperature, atmospheric pressure and humidity and non-weather factors, such as seasonal factors. The pollen concentration index is obtained by indexing a degree of possibility of occurrence of an allergic disease due to pollen. The pollen concentration index may be processed with daily average data of observation data by using pollen information of each region. The city high-temperature index is obtained by analyzing and modeling a relation between weather data and health data in order to provide a health damage forecast with respect to daily high-temperature dangerousness according to a weather forecast.

The life weather index may include a discomfort index, a heat index, an ultraviolet index, a sensory temperature index, a freeze and burst possibility index, and a food poisoning index. The life weather index class may infer a degree of danger with respect to each index and location.

The service ontology module 33 may support inference of a service suitable for a user by using health level information of a user and location danger degree information inferred through the internal ontology module 31 and the external ontology module 32. The service ontology module 33 may include an emergency class for treating an emergency situation, a GIS class for displaying a dangerous region according to a disease, and an exercise/food class for recommending an exercise and diet.

According to various embodiments of the present disclosure, the second electronic device 200 may include: the second communication module 290 that receives biological signals collected by the first sensor module 110 including the EMG sensor 112 and auxiliary sensor for detecting biological signals about a state of biological tissue of a wrist, the second display module 240 that outputs service information related to the received biological signals, and the second display control module 260 that controls the collection of the biological signals and the output of the service information.

According to various embodiments of the present disclosure, the biological signals may include biological signals about a state of at least one of extensor digitorum and extensor digiti minimi of a wrist and biological signals about a state of at least one of a radial artery and an ulnar artery of the wrist.

According to various embodiments of the present disclosure, the biological signals may include at least one of detection information of the acoustic vibration sensor 111, detection information of the optical sensor 113, detection information of the pressure sensor 115, detection information of the hall sensor 114 and detection information of the ECG sensor 117, which correspond to a state of vessels or muscles of a wrist, detection information of the taste sensor 121 for detecting a secretion of a user, and detection information of the olfactory sensor 123 for collecting smell information on a peripheral environment of the user.

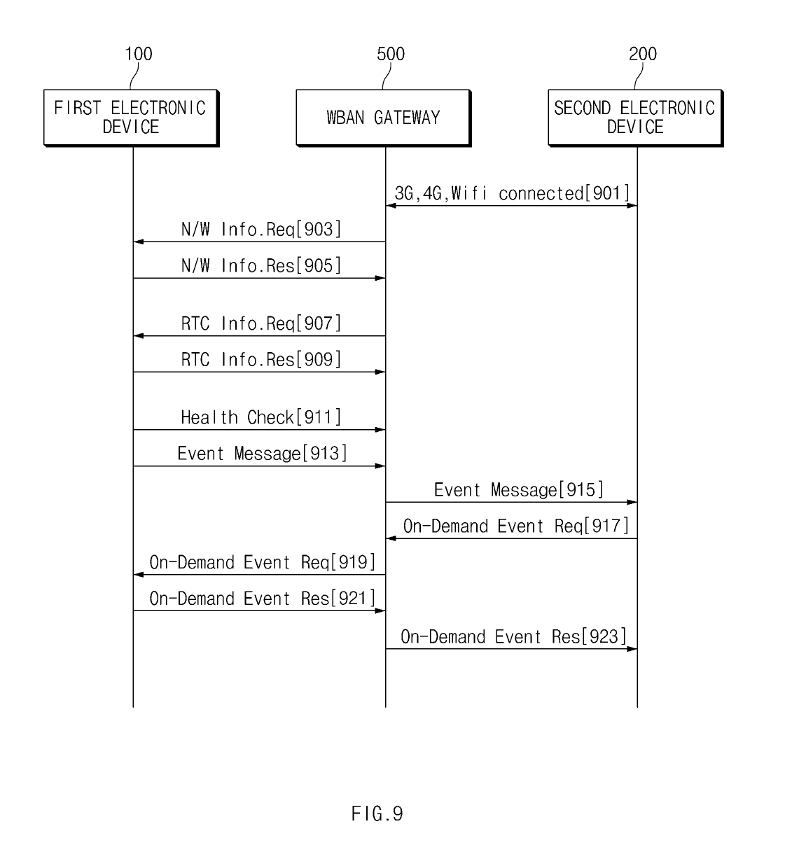

FIG. 9 is a flow diagram illustrating communication between a first electronic device and a second electronic device according to an embodiment of the present disclosure.

Referring to FIG. 9, it illustrates Wireless Body Area Network (WBAN) communication between the first electronic device 100 and the second electronic device 200 in order to describe communication between electronic devices. In operation 901, a WBAN gateway 500 and the second electronic device 200 may perform communication connection. In operation 903, the WBAN gateway 500 transmits an N/W Info.Req message to the first electronic device 100. In operation 905, the first electronic device 100 transmits an N/W infor.Res message to the WBAN gateway 500. In operation 907, the WBAN gateway 500 transmits a Real Time Clock (RTC) Info.Req message to the first electronic device 100. In operation 909, the first electronic device 100 transmits an RTC Info.Res message to the WBAN gateway 500. In operation 911, the first electronic device 100 transmits a health determination message to the WBAN gateway 500. In operation 913, the first electronic device 100 transmits an event message to the WBAN gateway 500. In operation 915, the WBAN gateway 500 transmits the event message to the second electronic device 200. In operation 917, the second electronic device 200 transmits an On-demand Event Req message to the WBAN gateway 500. In operation 919, the WBAN gateway 500 transmits the On-Demand Event Req message to the first electronic device 100. In operation 921, the first electronic device 100 transmits an On-demand Event Res message to the WBAN gateway 500. In operation 923, the WBAN gateway 500 transmits the On-Demand Event Res message to the second electronic device 200.

Through the above-mentioned communication operation, the first electronic device 100 may transfer biological signals to the second electronic device 200 and may receive service information corresponding to the biological signals from the second electronic device 200. The above-mentioned communication operation may be applied in the format of an Extensible Markup Language (XML) and Javascript Object Notation (JSON) according to which biological signal data received by the second electronic device 200 from the first electronic device 100 may be easily used in a web browser. According to the JSON format, which is a lightweight data structure format, a value is expressed with a pair of a key and a value, and repetitive operations are minimized to express data. When an amount of data is small, the JSON format may provide a higher processing speed than that of the XML format. In a general monitoring environment, such as a desktop computer, the second electronic device 200 and the first electronic device 100 may communicate with each other using the XML format. The biological signal value transmitted from the first electronic device to the second electronic device 200 can be transmitted to a cloud server device, and the WBAN gateway 500 may convert biological signal information into the XML format and JSON format so that the biological signal information is used in a general web environment. The N/W Info.Req is a network connection request message sent from the WBAN gateway 500 to the first electronic device 100 and the second electronic device 200. The N/W Info.Res is a response message for network configuration received by the WBAN gateway 500 from the first electronic device 100 and the second electronic device 200. The RTC Info.Req is an RTC initialization and response message. The RTC Info.Res is a message for transferring RTC information. The health determination is a message for periodically determining a state of connection to the first electronic device 100. The event message is a biological information message collected from the first electronic device 100 whenever an event occurs. The On-Demand Event Req is a biological information request message sent from the second electronic device 200 to a specific device. The On-Demand Event Res is a message for transferring biological information measured from the first electronic device 100.

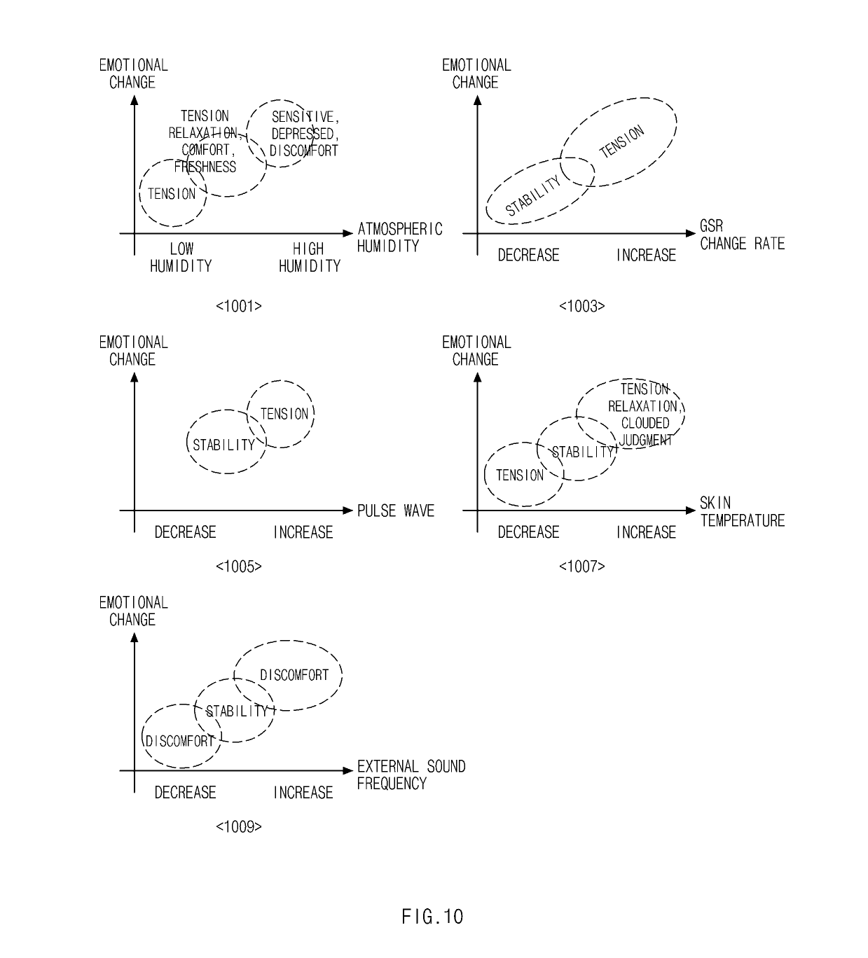

FIG. 10 is a diagram illustrating a biological signal-based service according to an embodiment of the present disclosure.

Referring to FIG. 10, the first electronic device 100 or the second electronic device 200 may Perform Biological Signal Recognition (PPG, oxygen saturation, pulse, and the like) and emotional recognition through Skin Resistance (GSR) and Skin Temperature (SKT). The biological signals may be generated during homeostatic maintenance, such as movement of an ion and transfer of action potential. The biological signals may be classified into electric signal, impedance signal, sound signals, biomagnetic signals, dynamic signals, chemical signals and optical signals. Such biological signals may be detected from any portion of a human body. The first electronic device 100 or the second electronic device 200 may detect a change of an emotion of a person due to a pulse wave that is a type of response of an autonomic nervous system, a human body temperature and skin resistance, an external temperature, external humidity and external noise.

A pulse wave (PPG) which shows physical characteristics of a heart is obtained by recording waveforms of changes of a pressure and pulse caused by blood that is pumped out from a heart and circulates in a body through arteries. A GSR is a type of an Electro Dermal Activity (EDA) indicating action of a sympathetic nervous system which is different depending on a portion of a skin by action of a parasympathetic nervous system. A change of the EDA is a biological electric phenomenon related to activity of an ANS. Therefore, the EDA may be used as a criterion on ANS activity. A current flow method or a potential method may be used to measure a skin resistance. A SKT greatly affects comfort and emotion. The SKT is measured over a human body surface in order to be applied to various fields. The SKT represents a temperature of a skin surface, more specifically, an outer shell surrounding a core, and is controlled by vasomotor activity. Expansion of a blood vessel increases a skin blood flow rate, but contraction of a blood vessel decreases the skin blood flow rate, causing a decrease of a skin temperature.

The first electronic device 100 or the second electronic device 200 may detect PPG, GSR and SKT signals and may extract emotional signals by measuring and synthetically analyzing a pulse, skin conductance and skin temperature, in order to infer a human emotion from biological signals caused by activity of an ANS of a human body. Accordingly, the first electronic device 100 or the second electronic device 200 may provide an emotional signal processing technology based on an emotional signal extraction function using a PPG sensor, an emotional signal extraction function using a GSR, an emotional signal extraction function using an SKT, a speed signal measurement and motion signal extraction function, a biological signal amplifying and noise filtering function, and an emotional state (arousal, neutral, relaxation) recognition function.

As illustrated in graphs 1001 to 1009, the first electronic device 100 or the second electronic device 200 may extract, from biological signals caused by activity of an autonomic nervous system, various values, e.g., changes of emotions, such as tension, relaxation of tension, comfort, freshness, sensitiveness, depression, discomfort and stability, according to atmospheric humidity in graph 1001, GSR change rate in graph 1003, pulse wave in graph 1005, SKT in graph 1007 and external sound frequency in graph 1009.

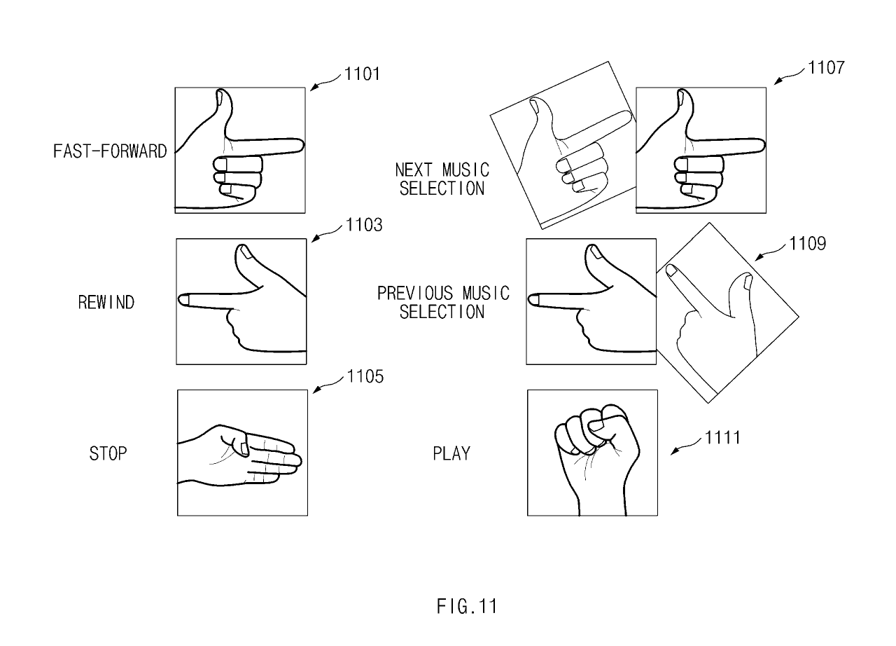

FIG. 11 is a diagram illustrating an operation of a biological signal-based device according to an embodiment of the present disclosure.

Referring to FIG. 11, the first electronic device 100 or the second electronic device 200 may support a control operation according to various hand motions related to playback of music. For example, as illustrated in graph 1101, when a user of the first electronic device 100 stretches a thumb and an index finger in a right direction while folding the other fingers, the first sensor module 110 may detect a corresponding muscle motion by using the EMG sensor 112 and at least one auxiliary sensor. Thereafter, the first electronic device 100 may perform a control operation so that music that is currently played or is standing by to be played is fast-forwarded.

As illustrated in graph 1103, when the user of the first electronic device 100 stretches a thumb and an index finger in a left direction while folding the other fingers, the first sensor module 110 may detect a corresponding muscle motion by using the EMG sensor 112 and at least one auxiliary sensor. Thereafter, the first electronic device 100 may perform a control operation so that music that is currently played or is standing by to be played is rewound.

As illustrated in graph 1105, when the user of the first electronic device 100 folds a thumb while stretching the other fingers, the first sensor module 110 may detect a corresponding muscle motion by using the EMG sensor 112 and at least one auxiliary sensor. Thereafter, the first electronic device 100 may perform a control operation so that music that is currently played is stopped.

As illustrated in graph 1107, when the user of the first electronic device 100 rotates a wrist by an angle of a certain degree from left to right while stretching a thumb and an index finger in a left direction and folding the other fingers, the first sensor module 110 may detect a corresponding muscle motion by using the EMG sensor 112 and at least one auxiliary sensor. Thereafter, the first electronic device 100 may perform a control operation so that next music is selected.

As illustrated in graph 1109, when the user of the first electronic device 100 rotates a wrist from right to left while stretching a thumb and an index finger in a left direction and folding the other fingers, the first sensor module 110 may detect a corresponding muscle motion by using the EMG sensor 112 and at least one auxiliary sensor. Thereafter, the first electronic device 100 may perform a control operation so that previous music is selected.

As illustrated in graph 1111, when the user of the first electronic device 100 makes a fist, the first sensor module 110 may detect a corresponding muscle motion by using the EMG sensor 112 and at least one auxiliary sensor. Thereafter, the first electronic device 100 may perform a control operation so that music that is stopped or is standing by to be played is played.

The first electronic device 100 may transfer biological signals corresponding to hand motions to the second electronic device 200 so that the music playback control operation described above with reference to graphs 1101 to 1111 is performed in the second electronic device 200. As described above, the first electronic device 100 or the second electronic device 200 may use the first electronic device 100 as an input interface. For example, the first electronic device 100 or the second electronic device 200 may support a character or number input function using a finger joint, a game controller function, a TV remote controller function, a media player operation control function through a gesture, a contactless three-dimensional multipoint (for example, iPointer, ETH: finger mouse) input function, and a sign language recognition function.

According to various embodiments of the present disclosure, when a user of the first electronic device 100 makes a specific motion, for example, when the user rapidly makes and opens a first three times and slowly makes and opens a first twice, and then makes and opens a first once again, the first electronic device 100 may request gesture unlocking from the second electronic device 200 while transferring biological signals corresponding to the motion to the second electronic device 200. The second electronic device 200 may perform gesture unlocking so as to be in a gesture process standby state, and may transfer a response to the request to the first electronic device 100. The first electronic device 100 may notify a gesture input standby state to the user through a vibration, information display, or audio output. When a gesture input of the user is detected, the first electronic device 100 may transfer a biological signal corresponding to the gesture input to the second electronic device 200, and the second electronic device 200 may determine a pattern of the biological signal so as to perform a process corresponding thereto.

When the transferred biological signal has a pattern corresponding to a gesture locking request, the second electronic device 200 may set a gesture locking state. The second electronic device 200 may notify the gesture locking state change to the first electronic device 100. The first electronic device 100 may output information related to gesture locking state notification and gesture standby end through a vibration, display module or audio module. According to various embodiments of the present disclosure, when a text message or a call is received, the first electronic device 100 may output a vibration for a corresponding state.

In order to separate the acceleration of gravity from the acceleration of motion and amplify a motion characteristic so as to recognize a hand motion state, the first electronic device 100 may calculate an arbitrary time point T-1, a time point T until which a certain period of time elapses from the arbitrary time point T-1, and a difference of characteristics corresponding to a window of the two time points, so as to use the calculated difference as a characteristic value of the window of the time point T. If a user directly uses average acceleration at the time point T as a characteristic when the first electronic device 100 stands still, values of three axes are, e.g., (x, y, z)=(0, 0, 9.8), but a difference between T-1 and T is calculated as (0, 0, 0). Therefore, there may be no detected motion.

In order to use the characteristic value as an input of a probability-based model, the first electronic device 100 performs symbolization (or quantization). Each symbol indicates an input node state of a probability network. After calculating characteristic values, the first electronic device 100 converts (or quantizes) each characteristic value into one of ten symbols by using a nonlinear analysis method. Since a gesture and a non-gesture have different characteristic value distributions, the first electronic device 100 performs nonlinear analysis to symbolize characteristic values. The first electronic device 100 extracts five reference values corresponding to upper 25%, upper 50%, upper 75% and a maximum value for each characteristic type by using training data corresponding to a non-gesture and a gesture. The extracted reference values concentrate at an interval where two models of non-gesture/gesture overlap each other, and thus, distinguishable symbols may be generated. Five non-gesture reference values and five gesture reference values (or 10 reference values in total) symbolize characteristic values nonlinearly. The converted 10 types of symbols are input as evidence values of the probability-based model. The first electronic device 100 applies, for example, a deep brief network (DBN) to a segmentation model and recognition model for recognizing a gesture.

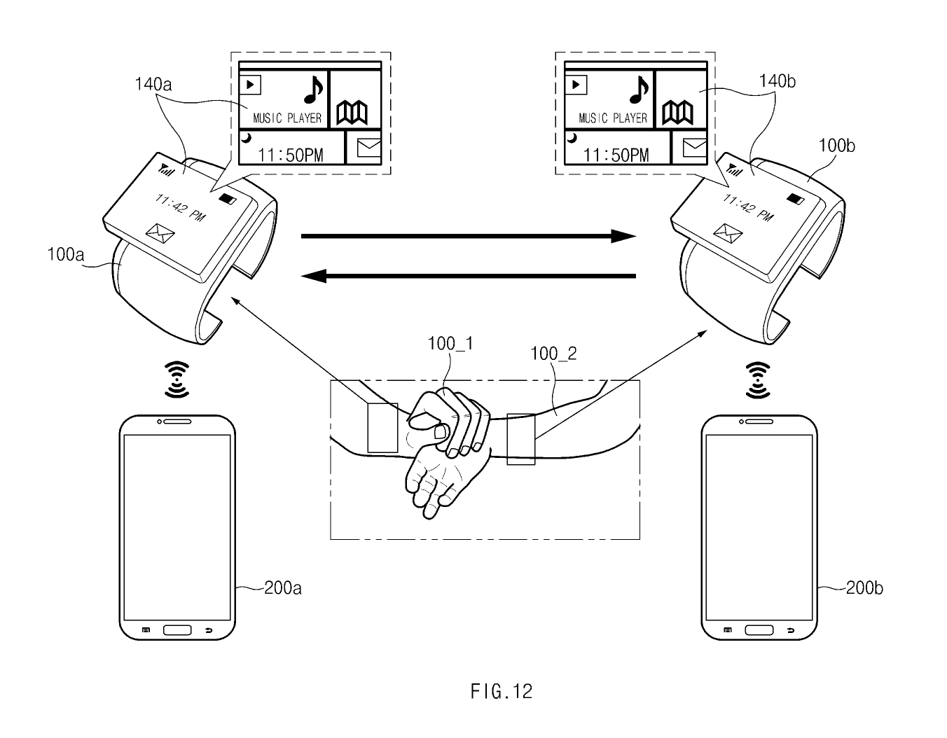

FIG. 12 is a diagram illustrating an operation of a biological signal-based service according to an embodiment of the present disclosure.

Referring to FIG. 12, a service operating system of the present disclosure may include an 11th electronic device 100a, a 21st electronic device 200a, a 12th electronic device 100b, and a 22nd electronic device 200b.

When users 100_1 and 100_2 contact with each other, the service operating system may support output of a sound according to the contact, based on the 11th electronic device 100a and 12th electronic device 100b which are watch-type biological recognition devices. When the first user 100_1 wearing the 11th electronic device 100a contacts with the second user 100_2 wearing the 12th electronic device 100b, the 11th electronic device 100a and the 12th electronic device 100b detect electron exchange through bodies of the first and second users 100_1 and 100_2. For example, the 11th electronic device 100a and the 12th electronic device 100b may measure a degree of skin reaction that generates a current. Thereafter, the degrees of skin reaction collected by the 11th electronic device 100a and 12th electronic device 100b may be uploaded to sound software of the 21st electronic device 200a and 22nd electronic device 200b. The information that is finally transferred to the sound software, i.e., the sound caused by the contact between the first and second users 100_1 and 100_2, may be output through the 21st electronic device 200a or 22nd electronic device 200b.

Processing of the sound caused by the contact between the first and second users 100_1 and 100_2 is processed in the 11th electronic device 100a and 12th electronic device 100b, and may be output through the 11th electronic device 100a and 12th electronic device 100b. During this process, information corresponding to the audio output caused by the contact between the first and second users 100_1 and 100_2 may be displayed on a display body 140a of the 11th electronic device 100a and a display body 140b of the 12th electronic device 100b.

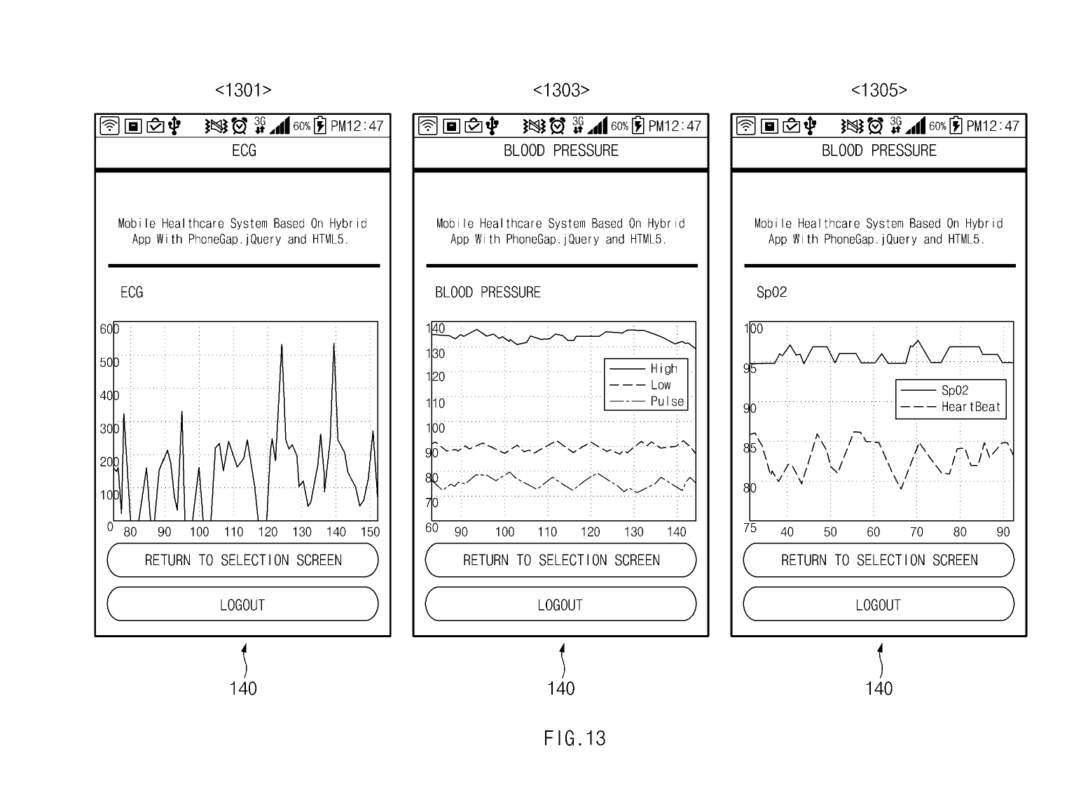

FIG. 13 is a diagram illustrating a screen interface provided during provision of a biological signal-based service according to an embodiment of the present disclosure.

Referring to FIG. 13, the first electronic device 100 may detect at least one of biological signals of ECG, blood pressure and Oxygen Saturation (SpO₂) by using the first sensor module 110. Furthermore, the first electronic device 100 may process the collected biological signals and may output, to the first display module 140, a corresponding screen interface, such as a screen 1301, a screen 1303 or a screen 1305. Here, the screen 1301 may be a screen for outputting a result of ECG detection. The screen 1303 may be a screen for outputting a result of blood pressure detection. The screen 1305 may be a screen for outputting a result of SpO₂ detection.

The first electronic device 100 may transfer the collected biological signals to the second electronic device 200. Therefore, the screen 1301, 1303 and 1305 may be screens output through the second display module 240 of the second electronic device 200.

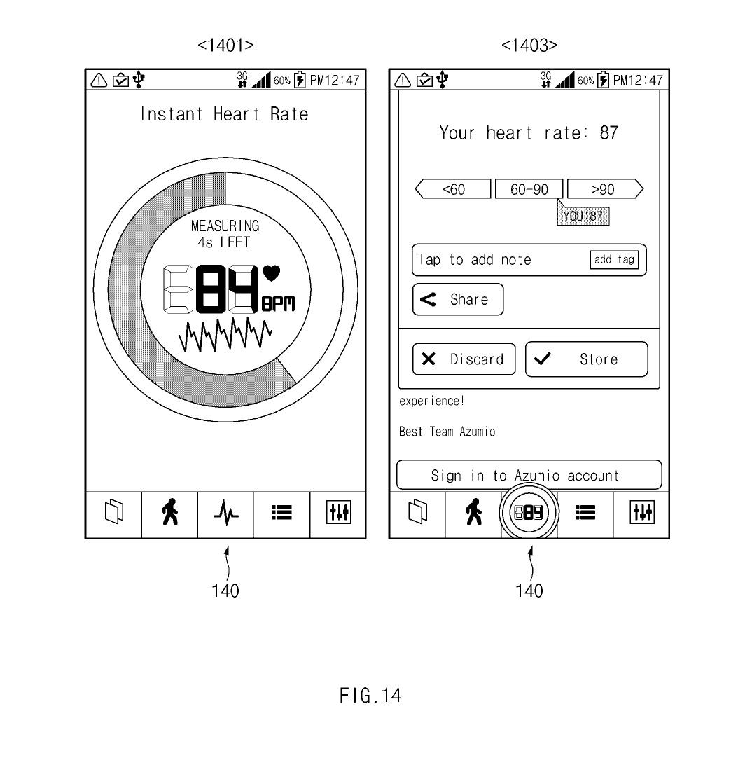

FIG. 14 is a diagram illustrating a screen interface provided during provision of a biological signal-based service according to an embodiment of the present disclosure.

Referring to FIG. 14, the first electronic device 100 may detect biological signals related to a heart rate. Here, the first electronic device 100 may detect motions of a radial artery and muscles adjacent thereto by using the EMG sensor 112 and the optical sensor 113. The first electronic device 100 may detect a heart rate based on the detected biological signals and may output, to the first display module 140, a screen interface in which graphic elements are mainly arranged as illustrated in a screen 1401. Alternatively, the first electron device 100 may output, to the first display module 140, a screen interface in which text elements are mainly arranged as illustrated in a screen 1403. The screen 1401 or screen 1403 may be adjusted according to selection by a user.

The first electronic device 100 may transfer heart rate information to the second electronic device 200. Therefore, the screens 1401 and 1403 may be output through the second display module 240.

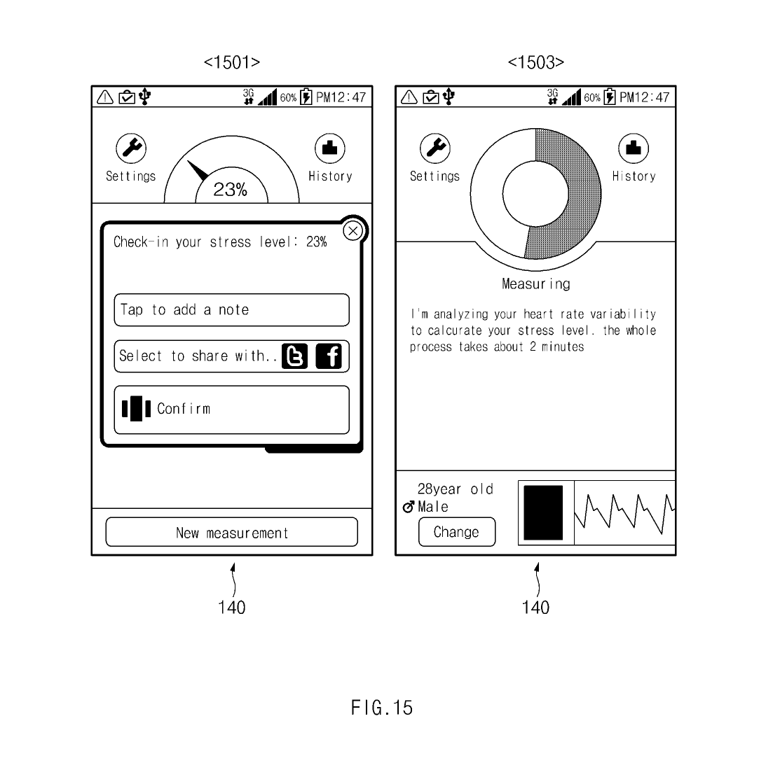

FIG. 15 is a diagram illustrating a screen interface provided during provision of a biological signal-based service according to an embodiment of the present disclosure.

Referring to FIG. 15, it illustrates an example of a screen interface for outputting stress index information of a user of the first electronic device 100. For example, a screen 1501 may be a screen interface in which a detected stress index of the user of the first electronic device 100 is provided mainly in the form of a text. A screen 1503 may be a screen interface in which a detected stress index of the user of the first electronic device 100 is provided mainly in the form of an image. The first electronic device 100 may output one of the screen 1501 or the screen 1503 related to the stress index through the first display module 140.

The first electronic device 100 may transfer the collected biological signals to the second electronic device 200 through the first sensor module 110. Therefore, the second electronic device 200 may detect the stress index of the user of the first electronic device 100 based on the received biological signals. Therefore, the second electronic device 200 may output the screen 1501 or screen 1503 related to the stress index of the user of the first electronic device 100 through the second display module 240.

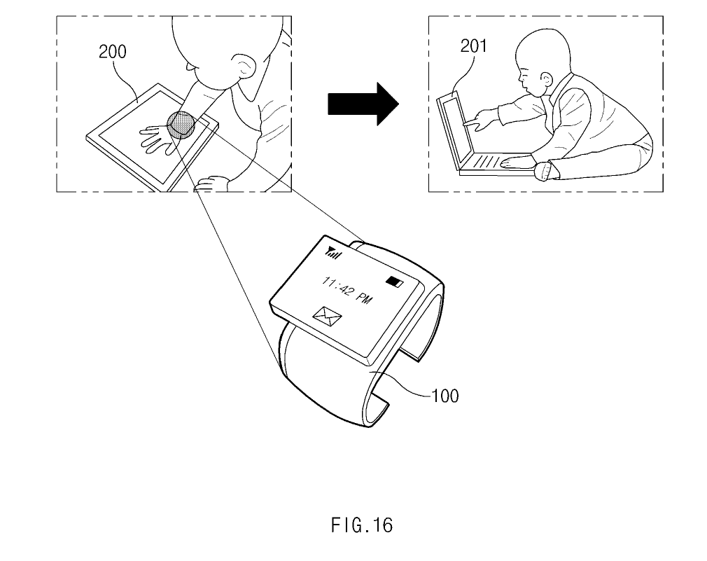

FIG. 16 is a diagram illustrating an operation of a service based on biological signals according to an embodiment of the present disclosure.

Referring to FIG. 16, a service operating system of the present disclosure may include the first electronic device 100, the second electronic device 200 and another electronic device 201.

The operation system having such a configuration may have a state that the first electronic device 100 that is a watch-type biological recognition device may be worn and operated by a user. For example, the first electronic device 100 may have a state capable of communicating data with the second electronic device 200 or other electronic devices 201 corresponding to the user input and schedule information after the user authentication. In this motion, the first electronic device 100 may support a function of transmitting data stored in the second electronic device 200 to the other electronic devices 201 when an electronic device recognition between heterogeneous devices (for example, the second electronic device 200 or other electronic device 201 recognition) occurs. For example, in the state that the user wears the first electronic device 100, the first electronic device 100 may establish a communication channel to the second electronic device 200, and perform a motion relating to a specific gesture input. When the designated gesture input event is generated, the first electronic device 100 may establish the communication channel to the second electronic device 200, and collect a file being output to a display module of the second electronic device 200 to store temporarily or semi-permanently. According to various embodiments of the present disclosure, the first electronic device 100 may collect a first sensor signal corresponding to a body reaction corresponding to the motion of catching a specific content displayed on the second electronic device 200, and transmit a message corresponding to the collected first sensor signal to the second electronic device 200. When the second electronic device 200 receives a message corresponding to the first sensor signal (for example, a biological signal corresponding to the motion of making a fist) designated from the first electronic device 100, the second electronic device 200 may transport at least one content output to the display module or stored in a storage module to the first electronic device 100 (the content is deleted in the second electronic 200 and is transferred to the first electronic device 100) or copy the content (is stored respectively in the first electronic device 100 and the second electronic device 200).

When a designated second sensor signal (for example, a biological signal corresponding to a motion of opening one's fist) collected, the first electronic device 100 may transmit a specific content to other electronic device 200. In this motion, the first electronic device 100 may automatically select the nearest neighbor other electronic device 200 and transmit the specific content to other electronic device 200. In addition, the first electronic device 100 may broadcast the specific content to at least one other electronic device 201 capable of communicating with the first electronic device 100 in the vicinity of the first electronic device 100. The first electronic device 100 may establish the communication channel to the other electronic device 200 in advance, or establish the communication channel to at least one other electronic device 200 by scanning the neighborhood at the time when the second sensor signal is generated. When the first electronic device 100 establishes the communication channel with the other electronic device 200, it may transmit at least one of content that is obtained from the second electronic device 200 to the other electronic device 201. The other electronic device 201 may output at least one received content to the display module. The first electronic device may delete the content that is transmitted to the other electronic device 201 from its storage module.

Here, at least one of the second electronic device 200 ant the other electronic device 201 may include at least one of a television, a Digital Video Disk player (DVD), an audio, a refrigerator, an air conditioner, an oven, a microwave, a washing machine, an air purifier, a set-top box, a TV box (for example, a SAMSUNG HomeSync™, an APPLE TV™, a GOOGLE TV™), a game console, an electronic dictionary, an electronic key, a camcorder, or a digital photo frame.

In relation to the above described function supporting, the electronic device 100 may supply at least one of a data transfer function or a data transfer mode. The electronic device 100 may examine whether it approaches the second electronic device 200 when the data transfer function or the dater transfer mode is activated. Further, the electronic device 100 may have a sensor signal receiving waiting (for example, a biological signal receiving waiting for opening or making a fist), or a sensor module activated state, and the like). The first electronic device 100 or the second electronic device 200 may identify a data transmit performing in a data transmitting mode or a proximity state using an IR sensor. The first electronic device 100 may perform transmitting data to the found other electronic device 201 using at least one communication method (for example, Near Field Communication (NFC), Bluetooth, WBAN or the like).

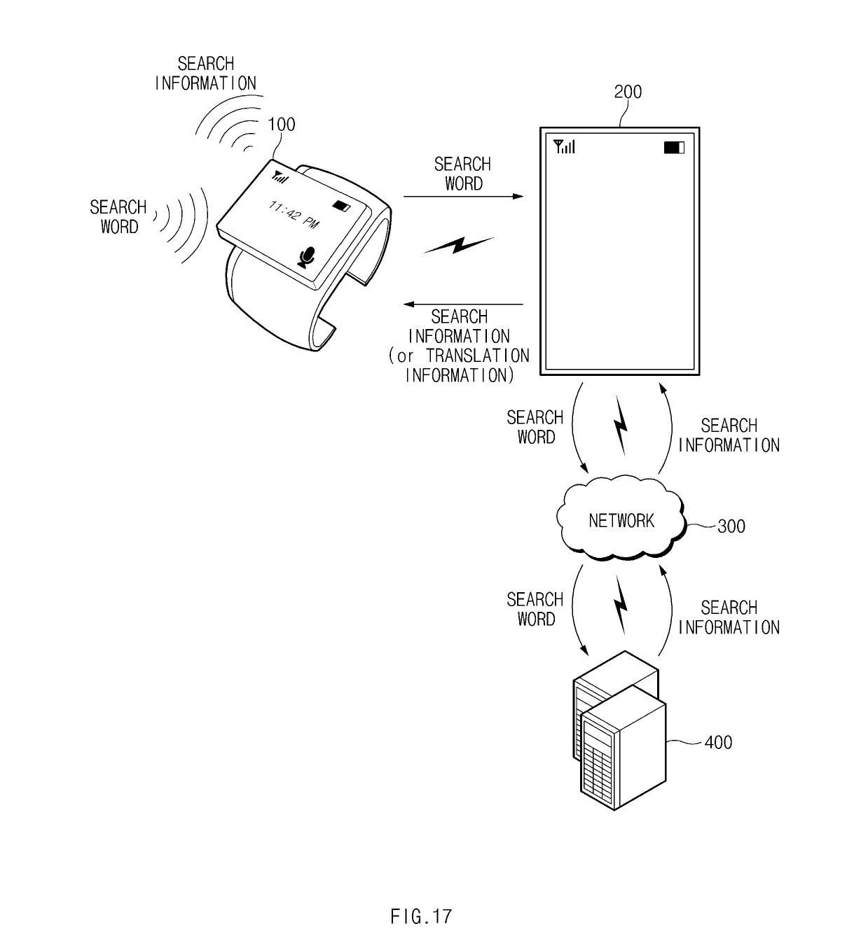

FIG. 17 is a diagram illustrating an operating of a service based on a biological signal according to an embodiment of the present disclosure.

Referring to FIG. 17, a first electronic device 100 may perform a result according to audio information processing using a second electronic device 200. For example, the first electronic device 100 may control to activate a MIC module corresponding to schedule information or a user input. The first electronic device 100 may receive voice (for example, a search word) information using the activated MIC module. The first electronic device 100 may transfer the received voice information to the second electronic device 200. In this motion, the first electronic device 100 may transmit the received analogue voice information to the second electronic device 200 as it is.

When the second electronic device 200 receives the search word (for example, at least one of analogue data or digital type recognition data), the electronic device 200 may perform a search information acquisition motion corresponding to the search word. For example, the second electronic device 200 may collect the search information relating to the search word of information stored in a storage module. According to various embodiments of the present disclosure, the second electronic device 200 may transfer the search word to a server device 400 of a designated specific web. In this motion, the second electronic device 200 may transfer the search word to the server device 400 using a network 300. The second electronic device 200 may transmit at least a part of the search information supplied by the server device 400 to the first electronic device 100. According to an embodiment of the present disclosure, the second electronic device 200 may select at least one of information of more than the designated confidence value among information that is supplied by the server device 400, and transmit the selected search information to the first electronic device 100. The second electronic device 200 may transmit the search information to the first electronic device 100 as a display data. According to various embodiments of the present disclosure, the second electronic device 200 may convert the search information to a voice signal and transmit the voice signal to the first electronic device 100.

The first electronic device 100 may receive the search information having at least one type of a display data type or a voice signal type that is received from the second electronic device 200. The first electronic device 100 may control the received search information to be output through at least one of the display module orb the audio module.

According to various embodiments of the present disclosure, the second electronic device 200 may obtain a translated data that is translated in a designated language corresponding to the search word received from the first electronic device 100. In this motion, the second electronic device may transmit the search word to the server device 400 supporting a translation function. When the second electronic device 200 receives the translated data (for example, at least one of a text type or a voice signal type) from the server device 400, it may transmit the translated data to the first electronic device 100. The first electronic device 100 may output the translated data to at least one of the display module or the audio module. According to various embodiments of the present disclosure, when the second electronic device 200 receives the translated data of the text type, it may convert the translated data of the text type to a voice signal to transmit the voice signal to the first electronic device 100.

According to an embodiment of the present disclosure, when “Sungnyemun” is input from the search word, the first electronic device 100 may transmit the voice signal or the recognition data to the second electronic device 200. The electronic device 200 may access to data network (3G, 4G or the like) and search data relating to Sungnyemun in the world wide web (www) environment (for example, an access environment of the server device 400).

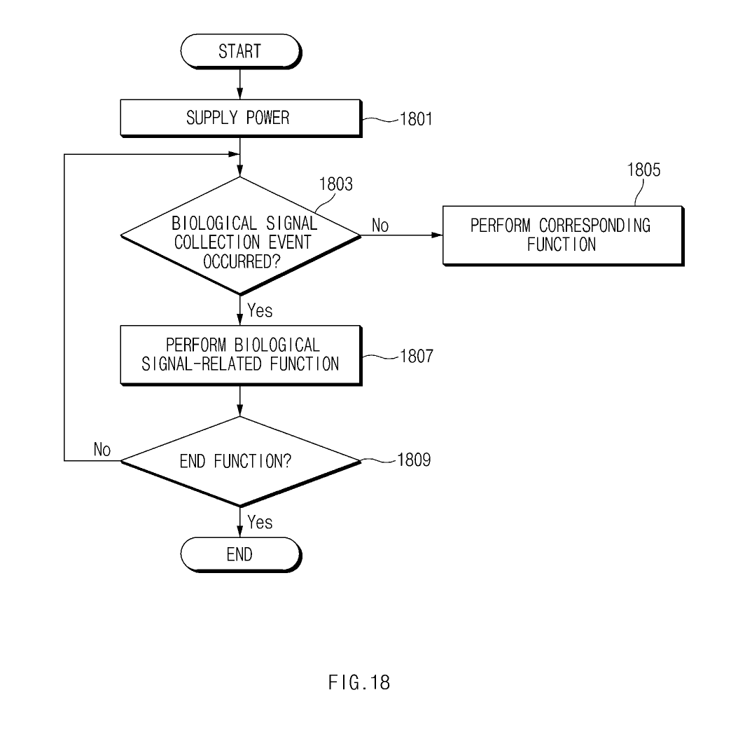

FIG. 18 is a flowchart illustrating a method of operating a first electronic device according to an embodiment of the present disclosure.

Referring to FIG. 18, in the method of operating of the first electronic device 100 of the present disclosure, it may be performed to supply electric power to the first electronic device 100 at an operation 1801. A first device control module 160 of the first electronic device 100 may identify whether a biological signal collecting event occurs in operation 1803. The first device control module 160 may activate at least one of a first sensor module 110 or a second sensor module 120 according to a set schedule or corresponding to an input event or a request of a second electronic device 200. In this regard, the first device control module 160 may output an icon or a menu item relating to the biological signal collecting.

In operation 1803, when a biological signal collecting events does not occur, the first device control module 160 may control a specific function to be performed according to the type of event in operation 1805. For example, the first device control module 160 may control a specific function that corresponds to an input event, such as a clock function, a call dialing function, a pedometer function, or the like, to be activated. Besides, the first device control module 160 may control the function that is being previously performed to be maintained.

When the biological signal collecting event occurs in operation 1803, the first device control module 160 may perform a function relating to the biological signal in operation 1807. According to an embodiment of the present disclosure, the first device control module 160 may receive the biological signal from the first sensor module 110 or the second sensor module 110 temporally, according to a regular interval, or in a real time. The first device control module 160 may analyze the received biological signal, and perform a biological signal related function according to the analyzed result. For example, the first device control module 160 may support performing functions, such as an active oxygen measurement function, a blood pressure detection function, a low blood pressure notifying function, a function of detecting and notifying signals of a brain hemorrhage and myocardial infarction, a blood vessel measurement function, a pulse wave stress measurement function, a medicine taking assistant function, a fall detection function, an action detection function, a heart rate measurement function, a living-along senior monitoring function, a snoring prevention function, a recipe providing function, a safe driving assistant or drowsy driving prevention function, a function of detecting an abnormal environment state using an olfactory sensor, a function of measuring sweat, moisture or secretion using a taste sensor, a business card recognition function (PAN), a bone conduction phone function, an anti-static function, a CAUEIS function, a biometric based emotion recognition service function, a Parkinson's disease diagnosis function, a lie detection function, a function of recognizing tennis swing using an acceleration sensor, a fieldwork function, a home network function, a U-themepark service function, a ComMotion function, a shopping assistant function, a cyber-guide function, a conference assistant function, a people & object pager function, and a function of data transfer of a host device by grap. Meanwhile, the first device control module 160 may transfer a collected biological signal in relation with supporting of the biological signal related function to the second electronic device 200. In this regard, the first device control module 160 may establish a communication channel to the second electronic device 200, and transmit the collected biological signal to the second electronic device 200. When the first device control module 160 receives biological signal related service information, it may output the biological signal related service information using at least one of the first display module 140, a vibration module 180 or the first audio module.

The first control module 160 may identify whether the event relating to a function termination occurs in operation 1809. When the function termination related event does not occur, the first device control module 160 may diverge into a procedure before the operation 1803 to support re-performing a subsequent procedure. Moreover, the first device control module 160 may diverge into a procedure before the procedure 1807 to support performing a specific biological signal related function. Meanwhile, when the function termination related event occurs, the first device control device 160 may control the biological signal related function to be terminated. Furthermore, the first device control module 160 may control a function that is performed before performing of the biological signal related function to be returned or control a standby screen state to be converted. According to an embodiment of the present disclosure, the first device control module 160 may support performing a basic function, such as performing a clock function.

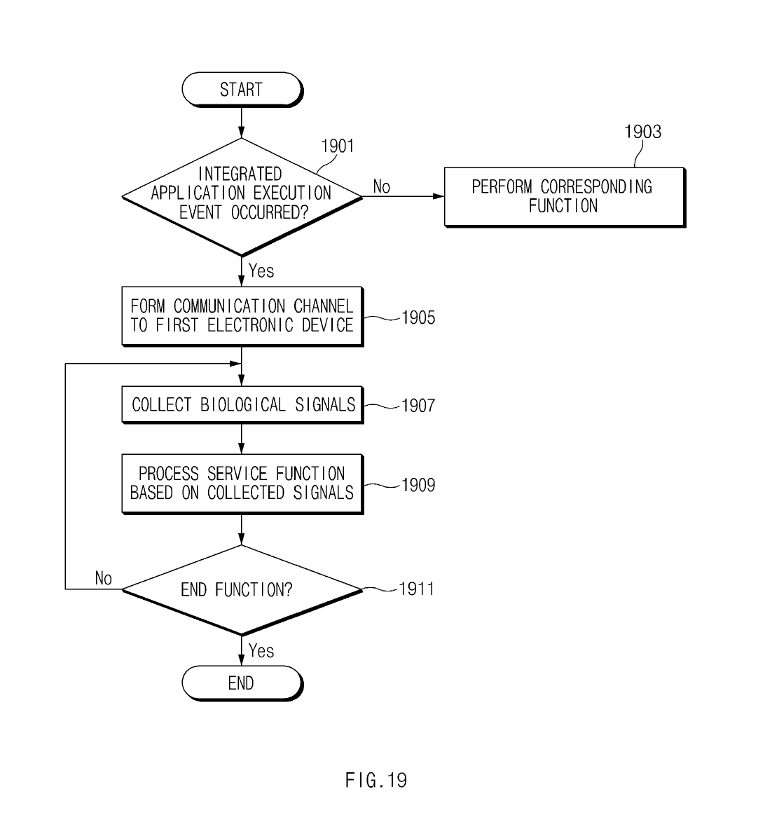

FIG. 19 is a diagram illustrating a method of operating a second electronic device according to an embodiment of the present disclosure.

Referring to FIG. 19, in the method of operating of the second electronic device, a second device control module 260 may identify whether an event relating to an execution of an integrated app 251 occurs in operation 1901. In this regard, the second device control module 260 may control a menu item relating to the execution of the integrated app operating 251 to be output. Meanwhile, the second device control module 260 may also receive an event that requires the execution of the integrated app 251 from the first electronic device. Moreover, the second device control module 260 may automatically execute the integrated app corresponding to a schedule event.

If the event that has occurred in operation 1901 is not related to the execution of the integrated app 251, the second device control module 260 may control performing a function corresponding to the event, such as a function of reproducing a specific sound source, a function of receiving a broadcast, a function of editing a file or the like in operation 1903. Meanwhile, the first device control module 260 may control the second electronic device 200 to be maintained in a previous state

When the event relating to the execution of the integrated app occurs in operation 1901, the second device control module 260 may perform establishing a communication channel to the first electronic device 100 in operation 1905. The second device control module 260 may receive a biological signal from the first electronic device 100 in operation 1907. In this operation, the second device control module 260 may require a specific biological signal of the first electronic device 100. Meanwhile, the second device control module 260 may receive a biological signal that is provided by the first electronic device 100, and activate the integrated app 251 corresponding to the biological signal.

The second device control module 260 may perform processing of a service function based on collecting of a signal using a collected biological signal in operation 1909. For example, the second device control module 260 may support performing functions, such as an active oxygen measurement function, a blood pressure detection function, a low blood pressure notifying function, a function of detecting and notifying signals of a brain hemorrhage and myocardial infarction, a blood vessel measurement function, a pulse wave stress measurement function, a medicine taking assistant function, a fall detection function, an action detection function, a heart rate measurement function, a living-along senior monitoring function, a snoring prevention function, a recipe providing function, a safe driving assistant or drowsy driving prevention function, a function of detecting an abnormal environment state using an olfactory sensor, a function of measuring sweat, moisture or secretion using a taste sensor, a business card recognition function (PAN), a bone conduction phone function, an anti-static function, a CAUEIS function, a biometric based emotion recognition service function, a Parkinson's disease diagnosis function, a lie detection function, a function of recognizing tennis swing using an acceleration sensor, a fieldwork function, a home network function, a U-themepark service function, a ComMotion function, a shopping assistant function, a cyber-guide function, a conference assistant function, a people & object pager function, and a function of data transfer of a host device by grap. The second device control module 260 may receive the biological signal from the electronic device 100, and support a specific function relating to the biological signal as based on the integrated app. In this operation, the second device control module 260 may perform accessing to a server device 400 through a communication network 300 when collecting of service information is needed, and receive specific service information from the server device 400. The second device control module 260 may transmit the received service information to the first electronic device 100.

The second device control module 260 may identify whether a function termination related event occurs in operation 1911. In this operation, when the function termination related event does not occur, the second device control module 260 may diverge into the operation before the operation 1907 and re-perform a subsequent operation or support performing the operation 1909. When the function termination related event does not occur, the second device control module 260 may suspend the execution of the integrated app. Furthermore, the second device control module 260 may control the communication channel to the first electronic device 100 to be released. The second device control module 260 may control the second electronic device 200 to be returned to a specific function that is previously performed or to be changed in a standby screen state, a lock screen display state, a sleep state or the like.

As described above, a function operating method based on biological signals and an electronic device supporting the same may collect a biological signal on a wrist based on an EMG sensor and an auxiliary sensor. As a result, the first electronic device 100 may perform a precise biological signal collecting. Furthermore, the first electronic device 100 may be prepared as a contact type on the wrist to more clearly detect the biological signal. Furthermore, the first electronic device 100 may have various sensors, and collect the compositive biological signals to support various biological signal related services based on the collected biological signals.

According to various embodiments of the present disclosure, the function operating method based on biological signals may include a procedure of collecting a biological signal according a state of a biological tissue using the EMG sensor 112 and the auxiliary sensor, a procedure of outputting information relating to the collected biological signal to a first display module 140 of the first electronic device 100.

According to various embodiments of the present disclosure, the procedure of collecting may include at least one of a procedure of collecting a biological signal corresponding to a schedule set in the first electronic device 100 or an input event or a procedure of receiving a biological signal collecting required from a second electronic device establishing a communication channel to the first electronic device 100.

According to various embodiments of the present disclosure, the procedure of collecting may include at least one of a procedure of detecting a state of at least one of extensor digitorum or extensor digiti minimi arranged on the wrist or a procedure of detecting a state of at least one of camellia radial or ulnar artery arranged on the wrist.

According to various embodiments of the present disclosure, the procedure of collecting may include a procedure of collecting at least one of detection information of the EMG sensor 112, detection information of an acoustic vibration sensor 111 detecting blood vessels or muscle arranged on the wrist, detection information of an optical sensor 113, detection information of a pressure sensor 115, detection information of a hole sensor 114, information of an ECG sensor 117, voice information, or detection information of an acceleration sensor 116 collecting a signal according a wrist movement.

According to various embodiments of the present disclosure, the method may further include at least one of a procedure of collecting detection information of a taste sensor 121 performing a detection of a wearer's secretion, or a procedure of collecting detection information of a nose sensor 123 collecting smell information of a wearer's environment.

According to various embodiments of the present disclosure, the method may further include at least one of a procedure of transmitting the collected biological signal to a second electronic device 200, or a procedure of receiving service information relating to the biological signal from the second electronic device 200. According to various embodiments of the present disclosure, the method may further include a procedure that the second electronic device 200 receives the biological signal, and a procedure of outputting service information relating to the received biological signal to a second display module 240 of the second electronic device 200.

The first storage module 150 or the second storage module 250 may include at least one of a volatile Memory (for example, a dynamic RAM(DRAM), a static RAM(SRAM) or a synchronous dynamic RAM(SDRAM) or the like), a non-volatile Memory (for example, one time programmable ROM(OTPROM), a programmable ROM(PROM), an erasable and programmable (ROM EPROM), an electrically erasable and programmable ROM(EEPROM), a mask ROM, a flash ROM, a NAND flash memory, or a NOR flash memory or the like).

According to an embodiment of the present disclosure, the first storage module 150 or the second storage module 250 may further include a Solid State Drive (SSID), a flash drive, for example, a Compact Flash (CF), a Secure Digital (SD), a Micro Secure Digital (Micro-SD), a Mini Secure Digital (Mini-SD), Extreme Digital (XD), a Memory stick or the like. Each of the above described elements of electrical devices according to the present disclosure may consist of one or more components, and terms of the elements may be varied according to types of electronic devices. The electronic device according to the present disclosure may consist of at least one of the above described elements, and part of elements may be omitted or additional other elements may be included. Furthermore, at least one of parts of elements of the electronic device according to the present disclosure is combined to configure an entity, thereby performing a same function of elements before configuring.

The term “module” may mean a unit including one or two more combinations. The “module may” be interchangeably used with the term “for example, a unit, a logic, a logical block, a component, or circuit or the like”. The “module” may become the smallest unit or part of the same performing one or more functions. The “module” may be electronically or mechanically implemented. For example, the “module” according to the present disclosure may include at least one of an Application-Specific Integrated Circuit (ASIC), Field-Programmable Gate Arrays (FPGAs), or a programmable-logic device that is already known, or developed in the future performing any motions.

According to various embodiments of the present disclosure, at least one part of devices (for example, modules or functions of the same) or methods (for example, motions) according to the present disclosure may be implemented in an instruction stored in a computer-readable storage media in a type of a programming module. When the instruction is performed by one or more processors, the one or more processors may perform a function corresponding to the instruction. At least a part of the programming modules may be implemented by the processor. At least a part of the programming module may include for example, a module, a program, a routine, sets of instructions, or a process or the like for performing one or more functions.

The computer-readable storage media may include Magnetic Medias, such as a hard disk, a floppy disk and a magnetic tape, Optical Medias, such as a Compact Disc Read Only Memory (CD-ROM), a Digital Versatile Disc (DVD), a Magneto-Optical Media, such as a Floptical Disk, and a Read Only Memory (ROM), Random Access Memory (ROM) and a flash memory and the like to be especially configured to store and perform a program instruction (for example, a programming module). Furthermore, the programming instruction may include a high level language a code capable of being performed through an interpreter by the computer as well as a machine code made by a compiler. The hardware device may be configured to operate as one or more software modules in order to perform the motion of the present disclosure, vice versa. The module or the programming module of the present disclosure may include at least one of the above described elements, or part of the elements may be omitted or additional other elements may be further included. Motions performed by the module, the programming module, or other elements according to the present disclosure may be performed in a sequential, parallel, iterative or heuristic manner. Furthermore, a part of motions may be performed in a different order, omitted, or include other motions.

According to a method and a device for operating electronic device functions based on biological signals, an electronic device is provided as a watch-type device so as to collect biological signals (EMG signal, flexor signal, extensor signal, acoustic vibration signal, and the like), and various electronic device functions may be supported based on the collected biological signals.

According to an embodiment of the present disclosure, various pieces of service information may be provided based on biological signals and peripheral environment information, and various electronic device functions are supported.

According to an embodiment of the present disclosure, motions of a finger, a hand or a wrist are detected based on a wrist wearable electronic device so that the device may be used as a menu input or control device.

According to an embodiment of the present disclosure, a specific situation may be notified to a user through information display, vibration or electrical stimulation of a wrist wearable electronic device.

According to an embodiment of the present disclosure, various pieces of biological information, such as oxygen saturation, pulse and stress index may be detected and notified to a user, and a U-health system may be established.

While the present disclosure has been shown and described with reference to various embodiments thereof, it will be understood by those skilled in the art that various changes in form and details may be made therein without departing from the spirit and scope of the present disclosure as defined by the appended claims and their equivalents.

What is claimed is:

1. An electronic device comprising:
a first sensor module comprising an Electromyogram (EMG) sensor configured to detect a state of a muscle of a wrist and an auxiliary sensor configured to detect biological signals about a state of biological tissue of the wrist;
a first display module configured to output information related to the biological signals collected by the first sensor module; and
a first device control module configured to control the first sensor module and the output of the information.

2. The electronic device of claim 1, wherein the first sensor module is further configured to detect a state of at least one of extensor digitorum or extensor digiti minimi of the wrist.

3. The electronic device of claim 1, wherein the first sensor module is further configured to detect a state of at least one of a radial artery or an ulnar artery of the wrist.

4. The electronic device of claim 1, wherein the auxiliary sensor comprises at least one of an acoustic vibration sensor, an optical sensor, a pressure sensor, a hall sensor or an Electrocardiogram (ECG) sensor configured to detect a state of a blood vessel or muscle of the wrist, and an acceleration sensor configured to collect a signal caused by a motion of the wrist.

5. The electronic device of claim 1, further comprising a second sensor module comprising at least one of a taste sensor configured to detect a secretion of a user and an olfactory sensor configured to collect smell information on a peripheral environment of the user.

6. The electronic device of claim 1, comprising:
a display body comprising the first display module and the first device control module; and
a connection body connected to both side of the display body,
wherein the first sensor module is arranged at a surface on an edge of the connection body connected to the display body, the surface facing the wrist.

7. The electronic device of claim 1, further comprising a first communication module configured to transmit the collected biological signals to a second electronic device.

8. The electronic device of claim 7, wherein the first communication module is further configured to receive service information related to the biological signals from the second electronic device.

9. The electronic device of claim 1, wherein the first display module is further configured to output at least one of information on an ECG, information on an heart rate, information on a blood oxygen saturation or blood pressure among the biological signals, and information on a stress index related to a pulse among the biological signals in the form of a text or image.

10. An electronic device comprising:
a second communication module configured to receive biological signals collected by a first sensor module comprising an Electromyogram (EMG) sensor and an auxiliary sensor configured to detect the biological signals about a state of biological tissue of a wrist;
a second display module configured to output service information related to the received biological signals; and
a second device control module configured to control the collection of the biological signals and the output of the service information.

11. The electronic device of claim 10, wherein the biological signals comprise biological signals about a state of at least one of extensor digitorum and extensor digiti minimi of the wrist and biological signals about a state of at least one of a radial artery and an ulnar artery of the wrist.

12. The electronic device of claim 10, wherein the biological signals comprise at least one of detection information of an acoustic vibration sensor, detection information of an optical sensor, detection information of a pressure sensor, detection information of a hall sensor, detection information of an Electrocardiogram (ECG) sensor, which correspond to a state of vessels or muscles of the wrist, detection information of a taste sensor for detecting a secretion of a user, and detection information of an olfactory sensor configured to collect smell information on a peripheral environment of the user.

13. A method for operating an electronic device, the method comprising:
collecting, by a first electronic device arranged on a wrist, biological signals about a state of biological tissue using an Electromyogram (EMG) sensor and an auxiliary sensor; and
outputting information related to the collected biological signals to a first display module of the first electronic device.

14. The method of claim 13, wherein the collecting of the biological signals about the state of the biological tissue comprises at least one of:
collecting the biological signals in response to a schedule event set in the first electronic device or an input event; and
receiving a biological signal collection request from a second electronic device that establishes a communication channel to the first electronic device.

15. The method of claim 13, wherein the collecting the biological signals about the state of the biological tissue comprises at least one of:
detecting a state of at least one of extensor digitorum and extensor digiti minimi of the wrist; and
detecting a state of at least one of a radial artery and an ulnar artery of the wrist.

16. The method of claim 13, wherein the collecting of the biological signals about the state of the biological tissue comprises:
collecting detection information of the EMG sensor; and
collecting at least one of detection information of an acoustic vibration sensor, detection information of an optical sensor, detection information of a pressure sensor, detection information of a hall sensor, detection information of an Electrocardiogram (ECG) sensor, which correspond to a state of vessels or muscles of the wrist, detection information of an acceleration sensor configured to collect a signal caused by a motion of the wrist, and detection voice information collected through voice recognition.

17. The method of claim 13, further comprising at least one of:
collecting detection information of a taste sensor configured to detect a secretion of a user; and
collecting detection information of an olfactory sensor configured to collect smell information on a peripheral environment of the user.

18. The method of claim 13, further comprising at least one of:
transmitting the collected biological signals to a second electronic device; and
receiving service information related to the biological signals from the second electronic device.

19. The method of claim 13, further comprising:
receiving, by a second electronic device, the biological signals; and
outputting, to a second display module of the second electronic device, service information related to the received biological signals.

20. The method of claim 19, wherein the outputting of the service information related to the received biological signals comprises outputting at least one of information on an ECG, information on an heart rate, information on an blood oxygen saturation or blood pressure among the biological signals, and information on a stress index related to a pulse among the biological signals in the form of a text or image.
